(19) United States
(12) Patent Application Publication
(10) Pub. No.: US 2009/0124587 A1
(43) Pub. Date: May 14, 2009

(54) METHODS FOR TREATING CANCER USING 17alpha-HYDROXYLASE/C17,20-LYASE INHIBITORS

(76) Inventors: Alan H. Auerbach, Hermosa Beach, CA (US); Arie S. Belldegrun, Los Angeles, CA (US)

Correspondence Address: JONES DAY, 222 EAST 41ST ST, NEW YORK, NY, 10017, US

(21) Appl. No.: 12/171,352

(22) Filed: Jul. 11, 2008

Related U.S. Application Data
(60) Provisional application No. 60/949,375, filed on Jul. 12, 2007.

(51) Int. Cl. A61K 31/58; A61P 35/00

(52) U.S. Cl. 514/176

(57) ABSTRACT

Methods for treating certain cancers in patients, such as mammals, using certain 17α-hydroxylase/C_{17,20}-lyase inhibitors are discussed herein. More particularly, methods for treating cancers comprising administering a 17α-hydroxylase/C_{17,20}-lyase inhibitor, such as abiraterone acetate (i.e. 3β-acetoxy-17-(3-pyridyl) androsta-5,16-diene) and metabolites thereof are described. In addition, methods for treating cancers that are refractory to hormone therapy or that are refractory to chemotherapy are also discussed. Finally novel dosing regimens and novel uses of the compounds discussed herein are disclosed.

This application claims priority to U.S. Provisional Application No. 60/949,375, filed on Jul. 12, 2007, which is incorporated herein by reference in its entirety.

### FIELD OF THE INVENTION

Methods for treating certain cancers in patients, such as mammals, using certain 17α-hydroxylase/C_{17,20}-lyase inhibitors are discussed herein. More particularly, methods for treating cancers comprising administering a 17α-hydroxylase/C_{17,20}-lyase inhibitor, such as abiraterone acetate (i.e. 3β-acetoxy-17-(3-pyridyl)androsta-5,16-diene) and metabolites thereof are described. In addition, methods for treating cancers that are refractory to hormone therapy or that are refractory to chemotherapy are also discussed. Finally, novel dosing regimens and novel uses of the compounds discussed herein are disclosed.

### BACKGROUND

Cancer is characterized by, inter alia, an increase in the number of abnormal cells derived from a given normal tissue, invasion of adjacent tissues by these abnormal cells, or lymphatic or blood-borne spread of malignant cells to regional lymph nodes and to distant sites (i.e., metastasis). Cancer is currently the second leading cause of death in the United States after heart disease. The number of people diagnosed with cancer has significantly increased and continues to rise at an alarming rate. Of special interest are individuals diagnosed with androgen-dependent disorders, such as prostate cancer, and estrogen-dependent disorders, such as breast, uterine, and ovarian cancer, since such cancers are among the most common and fatal forms of cancer and such diagnoses are rapidly increasing.

Prostate cancer is currently the most common non-skin cancer and the second leading cause of cancer-related death in men after lung cancer. The primary course of treatment for patients diagnosed with organ-confined prostate cancer is usually prostatectomy or radiotherapy. Not only are these treatments highly invasive and characterized by undesirable and serious side effects, such localized treatments are not effective on prostate cancer after it has metastasized. Moreover, surgery can be contraindicated due to the health of the patient or may be unacceptable to the patient, and a surgical procedure may not completely remove cancerous tissue. Radiation therapy is effective only when the irradiated neoplastic tissue exhibits a higher sensitivity to radiation than normal tissue. Furthermore, a large percent of individuals who receive localized treatments such as surgery or radiotherapy will suffer from recurring cancer and widespread metastases.

Additionally, breast cancer incidence in women has increased from one out of every 20 women in 1960 to one out of every eight women in 2005. Moreover, it is the most common cancer among white and African-American women. Similar to treating prostate cancer, most options for women diagnosed with breast cancer are highly invasive and have significant side-effects. Such treatments include surgery, radiation and chemotherapy. As with surgery and radiation therapies, there are many drawbacks to chemotherapy, including the fact that almost all chemotherapeutic agents are toxic, and chemotherapy causes significant, and often dangerous, side effects, including severe nausea, bone marrow depression, and immunosuppression. Additionally, many tumor cells are resistant or become resistant to chemotherapeutic agents through multi-drug resistance. Moreover, some patients for whom chemotherapy initially works may suffer from relapsing or recurring cancer.

Hormone therapy is another treatment option for individuals diagnosed with hormone-dependent, hormone-responsive, or hormone-sensitive cancers, such as prostate or breast cancer. Hormone therapy is a form of systemic treatment for cancers such as prostate or breast cancer wherein hormone ablation agents are used to suppress the production or block the effects of hormones, such as estrogen and progesterone in the body, which are believed to promote the growth of breast cancer, as well as, testosterone and dihydrotestosterone, which are believed to promote the growth of prostate cancer. Moreover, hormone therapy is less invasive than surgery and does not have many of the side effects associated with chemotherapy or radiation. Hormone therapy can also be used by itself or in addition to localized therapy and has shown to be effective in individuals whose cancer has metastasized.

Even though hormone therapy is less invasive and can be used on more advanced stages of cancer, some individuals administered current hormone therapy treatments may not show a significant response or may not show any response at all to such treatments. Additionally, as with chemotherapy, some patients treated with current hormone therapy treatments may suffer from relapsing or recurring cancer. Currently, such chemo-refractory and hormone-refractory cancer patients are left with very few treatment options.

Despite the progress made in the treatment of cancer there remains a need for more effective ways to treat cancer such as, but not limited to, prostate cancer and breast cancer. Additionally, there is a need for effective anti-cancer treatment options for patients who are not responding to current anti-cancer treatments, such as hormone therapy or chemotherapy. Also there is a need for effective anti-cancer treatment options for patients whose cancer has recurred.

### SUMMARY OF THE INVENTION

The present invention encompasses, among other things, methods for treating various types of cancer in a patient, e.g., a mammal, such as, but not limited to, hormone responsive cancers such as prostate or breast cancers. In certain embodiments provided herein is a method for the treatment of a cancer, such as in a patient that is refractory to a chemotherapy or a hormone therapy. In other embodiments, the patient is “chemotherapy-naïve.” In yet other embodiments, the patient is “hormone therapy-naïve.” In addition, provided herein are methods for reducing or avoiding the progress of metastases in patients having prostate or breast cancers. Further, described herein are methods of reducing plasma concentrations of prostate-specific antigen (PSA) in men.

The method comprises administering to a patient in need a therapeutically effective amount of a 17α-hydroxylase/C_{17,20}-lyase inhibitor. In some embodiments, the 17α-hydroxylase/C_{17,20}-lyase inhibitor is abiraterone acetate. Preferably, the 17α-hydroxylase/C_{17,20}-lyase inhibitor is administered with food to treat the diseases described herein. In certain embodiments, the therapeutically effective amount of a 17α-hydroxylase/C_{17,20}-lyase inhibitor can effect certain markers used to monitor a cancer in a patient such as the plasma concentration of PSA in prostate cancer patients. More specifically, the compounds discussed herein can be used to decrease PSA in various patients, including without limitation patients who are not castrated, patients who have been castrated, patients who have had their prostates removed, patients who have not had their prostates removed, patients who have received or are receiving chemotherapy, patients who are chemotherapy naïve, patients who have received or are receiving hormone therapy, or patients who are hormone therapy naïve.

Furthermore, in other embodiments, the methods of administering a 17α-hydroxylase/C_{17,20}-lyase inhibitor provides a maximum plasma concentration of the 17α-hydroxylase/C_{17,20}-lyase inhibitor or a metabolite thereof in a patient, such as a patient, at for example about 1 hour to about 8 hours after administration are described. In some embodiments, the methods of administering the 17α-hydroxylase/C_{17,20}-lyase results in maximum plasma concentration in a patient, such as a human, of, for example, from about 50 nmol/L to about 5000 nmol/l of the administered 17α-hydroxylase/C_{17,20}-lyase inhibitor or a metabolite thereof. Also, in some embodiments, the area under the curve of a plot of plasma concentration of the 17α-hydroxylase/C_{17,20}-lyase inhibitor or a metabolite of the administered compound in a patient versus time can range from about 50 hr*nmol/L to about 25,000 hr*nmol/L.

### DEFINITIONS

As used herein, and unless otherwise defined, the term “cancer,” refers to the growth, division or proliferation of abnormal cells in the body.

As used herein, and unless otherwise defined, the terms “treat,” “treating” and “treatment” include the eradication, removal, modification, management or control of a tumor or primary, regional, or metastatic cancer cells or tissue and the minimization or delay of the spread of cancer.

The term “17α-hydroxylase/C_{17,20}-lyase inhibitor” as used herein refers to an inhibitor of 17α-hydroxylase/C_{17,20}-lyase, an analog thereof, derivative thereof, metabolite thereof or pharmaceutically acceptable salt thereof. Also, unless otherwise noted, reference to a particular 17α-hydroxylase/C_{17,20}-lyase inhibitor can include analogs, derivatives, metabolites or pharmaceutically acceptable salts of such particular 17α-hydroxylase/C_{17,20}-lyase inhibitor.

As used herein, and unless otherwise defined, the term “patient” means an animal, including but not limited to an animal such as a human, monkey, cow, horse, sheep, pig, chicken, turkey, quail, cat, dog, mouse, rat, rabbit, or guinea pig. In one embodiment, the patient is a mammal and in another embodiment the patient is a human. In certain embodiments, the patient can be an adult male or female. In a particular embodiment, the patient has or is susceptible to having (e.g., through genetic or environmental factors) cancer. In a further embodiment, the patient has or is susceptible to having (e.g., through genetic or environmental factors) a tumor.

As used herein, and unless otherwise defined, the phrase “therapeutically effective amount” when used in connection with a 17-hydroxylase/C_{17,20}-lyase inhibitor means an amount of the 17α-hydroxylase/C_{17,20}-lyase inhibitor effective for treating a disease or disorder disclosed herein, such as cancer.

As used herein, and unless otherwise defined, the phrase “refractory cancer,” means cancer that is not responding to treatment or cancer that is not responding sufficiently to a treatment. Refractory cancer can also include recurring or relapsing cancer.

As used herein, and unless otherwise defined, the phrase “relapse cancer,” means cancer that was at one time responsive to a treatment but has returned or become no longer responsive to such treatment or is no longer responding sufficiently to such treatment.

As used herein, and unless otherwise defined, the phrase “recurring cancer,” means cancer that has returned after a patient has been earlier diagnosed with cancer, undergone treatment or had been previously diagnosed as cancer-free.

As used herein, and unless otherwise defined, the phrase “refractory patient,” means a patient who has refractory cancer.

As used herein, and unless otherwise defined, the phrases “hormone-refractory cancer” or “cancer that is refractory to hormone therapy” mean cancer that is not responding to a hormone therapy, cancer that is no longer responding to or has progressed through a hormone therapy, or cancer that is not responding sufficiently to a hormone therapy.

As used herein, and unless otherwise defined, the phrase “hormone-refractory patient,” means a patient who has hormone-refractory cancer.

As used herein, and unless otherwise defined, the phrases “chemo-refractory cancer” or “cancer that is refractory to chemotherapy” mean cancer that is not responding to chemotherapy, cancer that is no longer responding to or has progressed through chemotherapy, or cancer that is not responding sufficiently to chemotherapy.

As used herein, and unless otherwise defined, the phrase “chemo-refractory patient,” means a patient who has chemo-refractory cancer.

As used herein, and unless otherwise defined, the phrases “castration-refractory cancer” or “cancer that is refractory to castration” mean cancer that is not responding to castration, cancer that is no longer responding to or has progressed through castration, or cancer that is not responding sufficiently to castration.

As used herein, and unless otherwise defined, the phrases “chemotherapy-naïve patient” or “patient who is chemotherapy-naïve” refer to a patient who has never undergone chemotherapy treatment or who has never undergone chemotherapy treatment for a particular cancer.

As used herein, and unless otherwise defined, the phrases “hormone therapy-naïve patient” or “patient who is hormone therapy-naïve” refer to a patient who has never undergone hormone therapy treatment or who has never undergone hormone therapy treatment for a particular cancer.

The term “derivative” as used herein refers to a chemically modified compound wherein the chemical modification takes place at one or more functional groups of the compound. The derivative may retain or improve the pharmacological activity of the compound from which it is derived.

The term “analog” as used herein refers to a chemical compound that is structurally similar to another but differs slightly in composition (as in the replacement of one atom by an atom of a different element or in the presence of a particular functional group).

The phrase “pharmaceutically acceptable salt” as used herein refers to any salt of a 17α-hydroxylase/C_{17,20}-lyase inhibitor which may retain or improve the biological effectiveness of the 17α-hydroxylase/C_{17,20}-lyase inhibitor. Examples of pharmaceutically acceptable salts include, but are not limited to, acetates, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phylacetates, phenylpropionates, phenylbutyrates, citrates, lactates, gamma-hydroxybutyrates, glycollates, tartarates, alkanesulfonates (e.g. methane-sulfonate or mesylate), propanesulfonates, naphthalene-1-sulfonates, naphthalene-2-sulfonates, and mandelates. Several of the officially approved salts are listed in Remington: The Science and Practice of Pharmacy, Mack Publ. Co., Easton.

As used herein, and unless otherwise defined, the term “PSA” refers to prostate-specific antigen, which is a protein produced by the cells of the prostate gland.

As used herein, and unless otherwise defined, the term “castration levels of PSA” refers to the levels of PSA circulating in the blood of a patient who has had either one or both testicles surgically removed or has been chemically castrated, meaning the patient has been administered certain hormonal drugs to temporarily inhibit hormones that stimulate the testicles to produce testosterone.

As used herein, and unless otherwise defined, the term “castration levels of testosterone refers to the levels of testosterone circulating in the blood of a patient who has had either one or both testicles surgically removed or has been chemically castrated, meaning the patient has been administered certain hormonal drugs to temporarily inhibit hormones that stimulate the testicles to produce testosterone.

As used herein, the phrase “number of circulating tumor cells” refers to the number of cancer cells that have detached from a primary tumor and are circulating in the bloodstream or bone marrow of a cancer patient.

### BRIEF DESCRIPTION OF THE DRAWINGS

FIG. 1 is a plot of maximum plasma concentration as a function of administered dose of abiraterone acetate.

FIG. 2 is a plot of area under the curve (AUC) as a function of administered dose of abiraterone acetate.

FIG. 3 shows a pharmacokinetic profile for a first patient that was administered 1000 mg/day of abiraterone acetate.

FIG. 4 shows a pharmacokinetic profile for a second patient that was administered 1000 mg/day of abiraterone acetate.

FIG. 5 shows a pharmacokinetic profile for a third patient that was administered 1000 mg/day of abiraterone acetate.

FIG. 6 shows a pharmacokinetic profile for a fourth patient that was administered 1000 mg/day of abiraterone acetate.

FIG. 7 shows a pharmacokinetic profile for a fifth patient that was administered 1000 mg/day of abiraterone acetate.

FIG. 8 shows a pharmacokinetic profile for a sixth patient that was administered 1000 mg/day of abiraterone acetate.

FIGS. 9A-9D show the effect of the administration of abiraterone acetate on prostate-specific antigen (PSA) levels for subjects that were administered 250 mg/day, 500 mg/day, 750 mg/day and 1000 mg/day of abiraterone acetate.

FIG. 10 shows the regression of pelvic nodes in a subject that was administered 250 mg/day of abiraterone acetate.

FIG. 11 shows the regression of pelvic nodes in a subject that was administered 750 mg/day of abiraterone acetate.

FIG. 12 shows the regression of pelvic nodes in a subject that was administered 1000 mg/day of abiraterone acetate.

FIG. 13 shows the regression of mediastinal nodes in a subject that was administered 500 mg/day of abiraterone acetate.

FIG. 14 shows the regression of pulmonary metastases for a subject that was administered 750 mg/day of abiraterone acetate.

FIGS. 15A-15H show the blood plasma level of certain steroids over time in subjects that were administered 250 mg/day, 500 mg/day and 750 mg/day of abiraterone acetate.

### DETAILED DESCRIPTION OF THE INVENTION

Described herein are methods of treating cancer, particularly prostate and breast cancer, by administering to a patient, such as a mammal, preferably a human, a 17α-hydroxylase/C_{17,20}-lyase inhibitor.

Types of 17α-hydroxylase/C_{17,20}-lyase Inhibitors

Certain 17α-hydroxylase/C_{17,20}-lyase inhibitors are described in U.S. Pat. No. 5,604,213 to Barrie et al., which is herein incorporated by reference in its entirety. In certain embodiments, the 17α-hydroxylase/C_{17,20}-lyase inhibitor can be, without limitation, abiraterone or metabolites, analogs, derivatives or a pharmaceutical acceptable salts thereof. In some embodiments, the 17α-hydroxylase/C_{17,20}-lyase inhibitor can comprise 17-(3-pyridyl)androsta-5,16-dien-3β-ol; 17-(3-pyridyl)androsta-3,5,16-triene; 17-(3-pyridyl)androsta-4,16-dien-3-one; 17-(3-pyridyl)estra-1,3,5[10],16-tetraen-3-ol; 17-(3-pyridyl)-5α-androst-16-en-3α-ol; 17-(3-pyridyl)-5α-androst-16-en-3-one; 17-(3-pyridyl)-androsta-4,16-diene-3,11-dione; 17-(3-pyridyl)-androsta-3,5,16-trien-3-ol; 6α- and 6β-fluoro-17-(3-pyridyl)androsta-4,16-dien-3-one; 17-(3-pyridyl)androsta-4,16-dien-3,6-dione; 3α-trifluoromethyl-17-(3-pyridyl)androst-16-en-3β-ol or their acid addition salts and 3-esters as well as metabolites, analogs, derivatives or a pharmaceutical acceptable salts thereof.

In certain embodiments, the 17α-hydroxylase/C_{17,20}-lyase inhibitor can have the structure of formula (I):

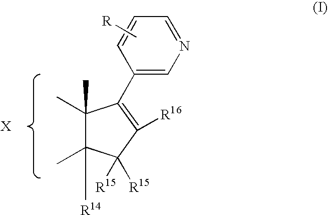

wherein X represents the residue of the A, B and C rings of a steroid which can be, without limitation, androstan-3α- or 3β-ol; androst-5-en-3α- or 3β-ol; androst-4-en-3-one; androst-2-ene; androst-4-ene; androst-5-ene; androsta-5,7-dien-3α or 3β-ol; androsta-1,4-dien-3-one; androsta-3,5-diene; androsta-3,5-diene-3-ol; estra-1,3,5[10]-triene; estra-1,3,5[10]-trien-3-ol; 5α-androstan-3-one; androst-4-ene-3,1-dione; 6-fluoroandrost-4-ene-3-one; or androstan-4-ene-3,6-dione; each of which, where structurally permissible, can be further derivatized in one or more of the following ways, including, but not limited to, to form 3-esters; to have one or more carbon or carbon ring double bonds in any of the 5,6-, 6,7-, 7,8-, 9,11- and 11,12-positions; as 3-oximes; as 3-methylenes; as 3-carboxylates; as 3-nitriles; as 3-nitros; as 3-desoxy derivatives; to have one or more hydroxy, halo, C₁₋₄-alkyl, trifluoro-methyl, C₁₋₄-alkoxy, C₁₋₄-alkanoyloxy, benzoyloxy, oxo, methylene or alkenyl substituents in the A, B, or C-ring; or to be 19-nor;

R represents a hydrogen atom or an alkyl group of 1-4 carbon atoms;

R¹⁴ represents a hydrogen atom, a halogen atom or an alkyl group of 1 to 4 carbon atoms;

each of the R¹⁵ substituents independently represents a hydrogen atom or an alkyl or alkoxy group of 1-4 carbon atoms, a hydroxy group or an alkylcarbonyloxy group of 2 to 5 carbon atoms or together represent an oxo or methylene group or R¹⁴ and one of the R¹⁵ groups together represent a double bond and the other R¹⁵ group represents a hydrogen atom or an alkyl group of 1 to 4 carbon atoms; and

R⁶ represents a hydrogen atom, halogen atom, or an alkyl group of 1 to 4 carbon atoms, in the form of the free bases or pharmaceutically acceptable acid addition salts, but excluding 3β-acetoxy-17-(3-pyridyl)androsta-5,14,16-triene, 3β,15α- and 3β,15β-diacetoxy-17-(3-pyridyl)androsta-5,16-diene and 3β-methoxy-17-(3-pyridyl-5α-androst-16-ene. Suitable inhibitors also include metabolites, derivatives, analogs, or pharmaceutically acceptable salts of formula (I).

In another embodiment, the 17-hydroxylase/C_{17,20}-lyase inhibitor can have the structure of formula (II):

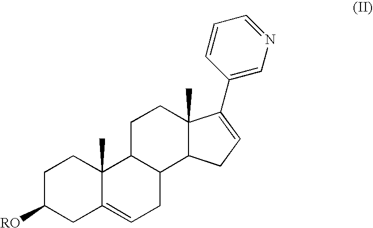

wherein R represents hydrogen or a lower acyl group having 1 to 4 carbons. Suitable inhibitors also include metabolites, derivatives, analogs, or pharmaceutically acceptable salts of formula (II).

In still another embodiment, the 17α-hydroxylase/C_{17,20}-lyase inhibitor can be a 3-alkanoyloxy-17-(3-pyridyl) androsta-5,16-diene in which the alkanoyloxy group has from 2 to 4 carbon atoms.

In a preferred embodiment, the 17α-hydroxylase/C_{17,20}-lyase inhibitor comprises abiraterone, or metabolites, derivatives, analogs and pharmaceutically acceptable salts thereof. Without being limited by any theory, abiraterone is believed to act by inhibiting the production of testosterone precursors by blocking the conversion of pregnenolone to deydroepiandrosterone (DHEA) and progesterone to androstenedione.

In one embodiment, a pharmaceutically acceptable salt of abiraterone is abiraterone acetate, or 3-acetoxy-17-(3-pyridyl)androsta-5,16-diene, which is the 3-acetate and a pro-drug form of abiraterone, and it has the following structural formula:

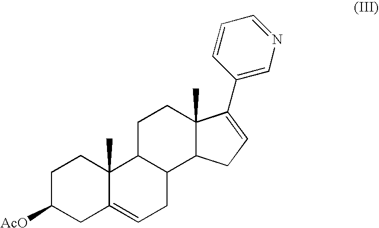

Preferred salts of abiraterone, such as abiraterone acetate, and methods of making such salts, are also disclosed in U.S. Provisional Application No. 60/603,559 to Hunt and U.S. patent application Ser. No. 11/660,869 to Hunt, which are incorporated by reference in their entirety. Preferred salts useful within the methods and compositions described herein include, but are not limited to, acetates, citrates, lactates, alkanesulfonates (e.g. methane-sulfonate or mesylate) and tartarates. Of special interest is abiraterone acetate mesylate salt (i.e. 3β-acetoxy-17-(3-pyridyl)androsta-5,16-diene mesylate salt) which has the following structural formula:

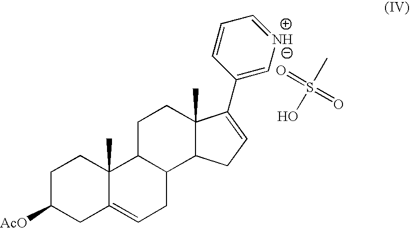

17α-hydroxylase/C_{17,20}-lyase inhibitors suitable for the methods and compositions described here can be made according to any method known to one skilled in the art. For example, such inhibitors can be synthesized according to the method disclosed in U.S. Pat. Nos. 5,604,213 and 5,618,807 to Barrie et al., herein incorporated by reference. Another method of making 17α-hydroxylase/C_{17,20}-lyase inhibitors is disclosed in PCT Publication No. WO 2006/021777 to Bury, herein incorporated by reference.

Dosages of the 17α-hydroxylase/C_{17,20}-lyase Inhibitor and Dosage Cycling

The therapeutically effective amounts or suitable dosages of the 17%-hydroxylase/C_{17,20}-lyase inhibitor depend upon a number of factors, including the nature of the severity of the condition to be treated, the particular inhibitor, the route of administration and the age, weight, and response of the individual patient. Suitable daily dosages of 17α-hydroxylase/C_{17,20}-lyase inhibitors can generally range, in single or divided or multiple doses, from about 10 mg/day to about 15,000 mg/day, about 10 mg/day to about 10,000 mg/day, about 10 mg/day to about 5,000 mg/day, about 10 mg/day to about 2,500 mg/day, about 10 mg/day to about 2,000 mg/day, about 10 mg/day to about 1,000 mg/day, from about 100 mg/day to about 15,000 mg/day, from about 100 mg/day to about 10,000 mg/day, from about 100 mg/day to about 5,000 mg/day, from about 100 mg/day to about 2,500 mg/day, from about 100 mg/day to about 2,000 mg/day, from about 100 mg/day to about 1,000 mg/day, from about 250 mg/day to about 15,000 mg/day, from about 250 mg/day to about 10,000 mg/day, from about 250 mg/day to about 5,000 mg/day, from about 250 mg/day to about 2,500 mg/day, from about 250 mg/day to about 2,000 mg/day, from about 250 mg/day to about 1,000 mg/day, from about 800 mg/day to about 15,000 mg/day, from about 800 mg/day to about 10,000 mg/day, from about 800 mg/day to about 5,000 mg/day, from about 800 mg/day to about 2,500 mg/day, from about 800 mg/day to about 2,000 mg/day, from about 1000 mg/day to about 15,000 mg/day, from about 11000 mg/day to about 10,000 mg/day, from about 1000 mg/day to about 5,000 mg/day, from about 1000 mg/day to about 2,500 mg/day, or from about 1000 mg/day to about 2,000 mg/day.

In some embodiments, the specific dosage of a 17α-hydroxylase/C_{17,20}-lyase inhibitor per day, in single or divided or multiple doses, by any route of administration (such as oral administration) includes without limitation about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1,000 mg, about 1,250 mg, about 1,500 mg, about 1,750 mg, about 2,000 mg, about 2,250 mg, about 2,500 mg about 2,750 mg, about 3,000 mg, about 3,250 mg, about 3,500 mg, about 3,750 mg, about 4,000 mg, about 4,250 mg, about 4,500 mg, about 4,750 mg, about 5,000 mg, about 5,250 mg, about 5,500 mg, about 5,750 mg, about 6,000 mg, about 6,250 mg, about 6,500 mg, about 6,750 mg, about 7,000 mg, about 7,250 mg, about 7,500 mg, about 7,750 mg, about 8,000 mg, about 8,250 mg, about 8,500 mg, about 8,750 mg, about 9,000 mg, about 9,250 mg, about 9,500 mg, about 9,750 mg, about 10,000 mg, about 10,250 mg, about 10,500 mg, about 10,750 mg, about 11,000 mg, about 11,250 mg, about 11,500 mg, about 11,750 mg, about 12,000 mg, about 12,250 mg, about 12,500 mg, about 12,750 mg, about 13,000 mg, about 13,250 mg, about 13,500 mg, about 13,750 mg, about 14,000 mg, about 14,250 mg, about 14,500 mg, about 14,750 mg, about 15,000 mg, about 15,250 mg, about 15,500 mg, about 15,750 mg, about 16,000 mg, about 17,000 mg, about 18,000 mg, about 19,000 mg, or about 20,000 mg.

Moreover, in some embodiments, the specific dosage of a 17α-hydroxylase/C_{17,20}-lyase inhibitor per day, in single or divided or multiple doses, by any route of administration (such as oral administration) includes without limitation about 10 mg or more, about 25 mg or more, about 50 mg or more, about 75 mg or more, about 100 mg or more, about 125 mg or more, about 150 mg or more, about 175 mg or more, about 200 mg or more, about 225 mg or more, about 250 mg or more, about 275 mg or more, about 300 mg or more, about 325 mg or more, about 350 mg or more, about 375 mg or more, about 400 mg or more, about 425 mg or more, about 450 mg or more, about 475 mg or more, about 500 mg or more, about 525 mg or more, about 550 mg or more, about 575 mg or more, about 600 mg or more, about 625 mg or more, about 650 mg or more, about 675 mg or more, about 700 mg or more, about 725 mg or more, about 750 mg or more, about 775 mg or more, about 800 mg or more, about 825 mg or more, about 850 mg or more, about 875 mg or more, about 900 mg or more, about 925 mg or more, about 950 mg or more, about 975 mg or more, about 1,000 mg or more, about 1,250 mg or more, about 1,500 mg or more, about 1,750 mg or more, about 2,000 mg or more, about 2,250 mg or more, about 2,500 mg or more, about 2,750 mg or more, about 3,000 mg or more, about 3,250 mg or more, about 3,500 mg or more, about 3,750 mg or more, about 4,000 mg or more, about 4,250 mg or more, about 4,500 mg or more, about 4,750 mg or more, about 5,000 mg or more, about 5,250 mg or more, about 5,500 mg or more, about 5,750 mg or more, about 6,000 mg or more, about 6,250 mg or more, about 6,500 mg or more, about 6,750 mg or more, about 7,000 mg or more, about 7,250 mg or more, about 7,500 mg or more, about 7,750 mg or more, about 8,000 mg or more, about 8,250 mg or more, about 8,500 mg or more, about 8,750 mg or more, about 9,000 mg or more, about 9,250 mg or more, about 9,500 mg or more, about 9,750 mg or more, about 10,000 mg or more, about 10,250 mg or more, about 10,500 mg or more, about 10,750 mg or more, about 11,000 mg or more, about 11,250 mg or more, about 11,500 mg or more, about 11,750 mg or more, about 12,000 mg or more, about 12,250 mg or more, about 12,500 mg or more, about 12,750 mg or more, about 13,000 mg or more, about 13,250 mg, or more about 13,500 mg or more, about 13,750 mg or more, about 14,000 mg or more, about 14,250 mg or more, about 14,500 mg or more, about 14,750 mg or more, about 15,000 mg or more, about 15,250 mg or more, about 15,500 mg or more, about 15,750 mg or more, about 16,000 mg or more, about 17,000 mg or more, about 18,000 mg or more, about 19,000 mg or more, or about 20,000 mg or more.

Also, in some embodiments, the therapeutically effective amount of the 17α-hydroxylase/C_{17,20}-lyase inhibitor may be administered once per day. In other embodiments, the 17α-hydroxylase/C_{17,20}-lyase inhibitor is administered more than once per day. Also, the frequency in which any of these inhibitors can be administered can be once or more than once, (e.g. twice, 3 times, 4 times, etc.) per about 2 days, about 3 days, about 4 days, about 5 days, about 6 days, about 10 days, about 20 days, about 28 days, about a week, about 2 weeks, about 3 weeks, about 4 weeks, about a month, about every 2 months, about every 3 months, about every 4 months, about every 5 months, about every 6 months, about every 7 months, about every 8 months, about every 9 months, about every 10 months, about every 11 months, about every 12 months, about every year, about every 2 years, about every 3 years, about every 4 years, or about every 5 years.

Furthermore, the above frequencies of administration can occur continuously or non-continuously over certain time periods. For example, a certain amount of a 17α-hydroxylase/C_{17,20}-lyase inhibitor can be administered daily continuously over 28 days. Time periods over which the frequencies of administration can occur continuously or non-continuously include without limitation about 1 day, about 2 days, about 3 days, about 4 days, about 5 days, about 6 days, about 10 days, about 20 days, about 28 days, about a week, about 2 weeks, about 3 weeks, about 4 weeks, about a month, about every 2 months, about every 3 months, about every 4 months, about every 5 months, about every 6 months, about every 7 months, about every 8 months, about every 9 months, about every 10 months, about every 11 months, about every 12 months, about every year, about every 2 years, about every 3 years, about every 4 years, or about every 5 years.

In some embodiments, the therapeutically effective amount of the 17α-hydroxylase/C_{17,20}-lyase inhibitor is administered using dose cycling or a dosing regimen in which the 17α-hydroxylase/C_{17,20}-lyase inhibitor is administered at a certain frequency, such as those discussed above, during a certain treatment period of a particular time duration, such as those described above. The treatment period is then followed by a non-treatment period of a certain time duration, such as the time periods described above, in which the 17α-hydroxylase/C_{17,20}-lyase inhibitor is not administered to the patient. In certain embodiments, no 17α-hydroxylase/C_{17,20}-lyase inhibitor is administered during the non-treatment period. In other embodiments, another 17α-hydroxylase/C_{17,20}-lyase inhibitor is administered during the non-treatment period.

This non-treatment period can then be followed by a series of subsequent treatment and non-treatment periods of the same or different frequencies or the same or different lengths of time. In some embodiments, the treatment and non-treatment periods are alternated. In other embodiments, a first treatment period in which a first amount of the 17α-hydroxylase/C_{17,20}-lyase inhibitor is administered can be followed by another treatment period in which a same or different amount of the same or a different 17α-hydroxylase/C_{17,20}-lyase inhibitor is administered. The second treatment period can be followed by other treatment period. During the treatment and non-treatment periods, one or more additional therapeutic agents can be administered to the patient.

Methods and Timing of Administration of the 17α-hydroxylase/C_{17,20}-lyase Inhibitor

The 17α-hydroxylase/C_{17,20}-lyase inhibitor can be administered by any method known to one skilled in the art. The 17α-hydroxylase/C_{17,20}-lyase inhibitor can be administered in the form of a composition, in one embodiment a pharmaceutical composition, such as those described below. Preferably the composition containing the 17α-hydroxylase/C_{17,20}-lyase inhibitor is pharmaceutically suitable for oral administration.

Examples of modes of administration include parenteral (e.g., subcutaneous, intramuscular, intraorbital, intracapsular, intraspinal, intrasternal, intravenous, intradermal, intraperitoneal, intraportal, intra-arterial, intrathecal, transmucosal, intra-articular, and intrapleural), transdermal (e.g., topical), epidural, and mucosal (e.g., intranasal) injection or infusion, as well as oral, inhalation, pulmonary, and rectal administration.

The 17-hydroxylase/C_{17,20}-lyase inhibitor can be administered at various times during the course of the day, e.g., in the morning or in the evening. In some embodiments, the 17α-hydroxylase/C_{17,20}-lyase inhibitor is administered with food. This means that the 17α-hydroxylase/C_{17,20}-lyase inhibitor is taken by the patient while ingesting food, immediately after consumption of food by the patient, or immediately before consumption of food by the patient. In other embodiments, the inhibitor is administered about 1 minute to about 1 hour after consumption of food by the patient. In other embodiments, the 17α-hydroxylase/C_{17,20}-lyase inhibitor is administered about 1 minute to about 30 minutes after consumption of food by the patient. The 17α-hydroxylase/C_{17,20}-lyase inhibitor can be administered with food at the frequencies and over the same time periods as discussed above. Also, the 17α-hydroxylase/C_{17,20}-lyase inhibitor can be administered with food in a dosing regimen such as those described above. For example, in the one embodiment, the 17α-hydroxylase/C_{17,20}-lyase inhibitor is administered once per day with food continuously during a first treatment cycle of about 28 days.

Alternatively, the 17α-hydroxylase/C_{17,20}-lyase inhibitor can also be administered during periods of fasting. In some embodiments, the 17α-hydroxylase/C_{17,20}-lyase inhibitor is administered first thing in the morning, before any food has been consumed by the patient. In certain embodiments, the inhibitor is administered after the patient has fasted for less than about 5 hours. In other embodiments, the 17α-hydroxylase/C_{17,20}-lyase inhibitor is administered after the patient has fasted for less than about 2 hours. The 17α-hydroxylase/C_{17,20}-lyase inhibitor can be administered after fasting at the frequencies and over the same time periods as discussed above. Furthermore, the 17α-hydroxylase/C_{17,20}-lyase inhibitor can be administered after fasting and during a dosing regimen such as those described above.

Compositions Containing a 17α-hydroxylase/C_{17,20}-lyase Inhibitor

In certain embodiments, the compositions contain a 17α-hydroxylase/C_{17,20}-lyase inhibitor, preferably abiraterone acetate. Such compositions can take various forms such as, but not limited to, solutions, suspensions, emulsions, tablets, pills, capsules, powders or sustained-release formulations, depending on the intended route of administration.

For topical or transdermal administration, the compositions can be formulated as solutions, gels, ointments, creams, suspensions or salves.

For oral administration, the compositions may be formulated as tablets, pills, dragees, troches, capsules, liquids, gels, syrups, slurries, suspensions and the like, for oral ingestion by a patient to be treated.

The composition may also be formulated in rectal or vaginal compositions such as suppositories or retention enemas that contain conventional suppository bases such as cocoa butter or other glycerides.

In addition to the formulations described previously, the composition may also be formulated as a depot preparation. Such long acting formulations may be administered by implantation (for example subcutaneously or intramuscularly) or by intramuscular injection. Thus, for example, the therapeutic agents may be formulated with suitable polymeric or hydrophobic materials (for example as an emulsion in an acceptable oil) or ion exchange resins, or as sparingly soluble derivatives, for example, as a sparingly soluble salt.

Additionally, the composition may be delivered using a sustained-release system, such as semi-permeable matrices of solid polymers containing the composition. Various forms of sustained-release materials have been established and are well known by those skilled in the art. Sustained-release capsules, depending on their chemical nature, can release the composition over a period of hours, days, weeks, or months. For example, a sustained release capsule can release the compositions over a period of 100 days or longer. Depending on the chemical nature and the biological stability of the composition, additional strategies for stabilization may be employed.

The compositions can further comprise a pharmaceutically acceptable carrier. The term “carrier” refers to a diluent, adjuvant (e.g., Freund's adjuvant (complete and incomplete)), excipient, or vehicle with which the therapeutic is administered.

For parenteral administrations, the composition can comprise one or more of the following carriers: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampules, disposable syringes or multiple dose vials made of glass or plastic.

For oral solid formulations suitable carriers include fillers such as sugars, e.g., lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, fats and oils; granulating agents; and binding agents such as microcrystalline cellulose, gum tragacanth or gelatin; disintegrating agents, such as cross-linked polyvinylpyrrolidone, agar, or alginic acid or a salt thereof such as sodium alginate, Primogel, or corn starch; lubricants, such as magnesium stearate or Sterotes; glidants, such as colloidal silicon dioxide; a sweetening agent, such as sucrose or saccharin; or flavoring agents, such as peppermint, methyl salicylate, or orange flavoring. If desired, solid dosage forms may be sugar-coated or enteric-coated using standard techniques.

For intravenous administration, suitable carriers include physiological saline, bacteriostatic water, phosphate buffered saline (PBS). In all cases, the composition must be sterile and should be fluid to the extent that easy injectability with a syringe. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars; polyalcohols such as mannitol, sorbitol; sodium chloride in the composition. Prolonged absorption of the injectable compositions can be brought about by including in the composition an agent which delays absorption, for example, aluminum monostearate and gelatin.

Also for intravenous administration, the compositions may be formulated in solutions, preferably in physiologically compatible buffers such as Hanks's solution, Ringer's solution, or physiological saline buffer. The solution may contain formulatory agents such as suspending, stabilizing and/or dispersing agents. In a preferred embodiment, the compositions are formulated in sterile solutions.

For transmucosal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories.

For administration by inhalation, the compositions may be formulated as an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of gelatin for use in an inhaler or insulator may be formulated containing a powder mix of the composition and a suitable powder base such as lactose or starch.

The pharmaceutical compositions may be manufactured by means of conventional mixing, dissolving, granulating, dragee-making, levigating, emulsifying, encapsulating, entrapping or lyophilizing processes.

### Cancers to be Treated and Patient Population

Cancers that can be treated with the methods and compositions described herein include, but are not limited to, those cancers that are hormone-dependent, hormone responsive, or hormone sensitive (collectively, “hormone responsive cancers”). In particular, cancers that can be treated include, without limitation, androgen-responsive cancers, such as testosterone-responsive cancers, and estrogen-responsive cancers. Moreover, in some embodiments, the cancer can be a non-hormone-responsive cancer or a cancer that was hormone responsive but later becomes non-hormone responsive. Also, cancers that can be treated include, without limitation, prostate cancer, breast cancer, testicular cancer, penile cancer, vaginal cancer, vulvar cancer, cervical cancer, uterine cancer, ovarian cancer, cancer of the Fallopian tubes or other reproductive organ cancers. In other embodiments, cancers that may be treated include, without limitation, adrenal cancer, cancer of the lymphatic system, such as the lymph nodes, leukemia, lymphoma, myeloma, Waldenström's macroglobulinemia, monoclonal gammopathy, benign monoclonal gammopathy, heavy chain disease, bone and connective tissue sarcoma, brain tumors, thyroid cancer, pancreatic cancer, pituitary cancer, eye cancer, esophageal cancer, stomach cancer, colon cancer, rectal cancer, liver cancer, gallbladder cancer, cholangiocarcinoma, lung cancer, oral cancer, skin cancer, kidney cancers, Wilms' tumor and bladder cancer.

In addition, the methods and compositions described herein can be used to treat various stages of a particular cancer or specific forms of a type of cancer, such as breast or prostate cancer. In some embodiments, the methods and compositions can be used to treat a pre-cancerous state, a post cancerous state, an advanced cancerous state, a pre-metastatic cancer, a cancer that is a metastasis, i.e. has metastasized from another cancer, a cancer that has metastasized to, for example, the bones, lymph nodes, lungs, or a non-metastatic cancer. In one embodiment, the patients treated are newly diagnosed with cancer, such as prostate cancer. In embodiments relating to prostate cancer, the prostate cancer may be non-metastatic; may be pre-metastatic; may have metastasized to the bones, lungs, lymph nodes; or may comprise advanced adenocarcinoma of the prostate. For example, the prostate cancer can be, without limitation, androgen-dependent prostate cancer; androgen-independent prostate cancer (AIPC) such as those that are biochemical-only in nature, asymptomatic with positive scans or symptomatic. The stages of prostate cancer that may be treated by the methods and compositions described herein include, without limitation, stages IA, IIB, II, III, IVA, IVB, and IVC; cancers with a Gleason grade of between 1 and 5 or a Gleason sum or score of between 2 and 10; stages A, B, C, or D under the Jewett-Whitmore system; and any combination of TNM scores under the American Joint Committee on Cancer (AJCC) TNM system, including, without limitation, TX, T0, T1ₐ, T1_{b}, T2ₐ, T2_{b}, T3ₐ, T3_{b}, T4, NX, N0, N1, N2, N3, MX, M0, M1ₐ, M1_{b}, M1_{c}.

Furthermore, the methods and compositions described herein can be used to treat non-cancerous or pre-cancerous conditions, such as but not limited to those that relate to PSA. For example, such conditions include without limitation prostatitis (inflammation of the prostate), such as acute bacterial prostatitis, acute urinary retention, asymptomatic inflammatory prostatitis, chronic bacterial prostatitis, or chronic nonbacterial prostatitis; benign prostatic hyperplasia (BPH) (enlargement of the prostate) or prostatic intraepithelial neoplasia (PIN).

Patients who may be treated with the methods described herein include mammals. In preferred embodiments, the patient is a human. The patient can be female or male. The patient may have a family history of the same or a different cancer. For example, the patient can be a female who has breast or ovarian cancer and has one or more family members who have or have had breast or ovarian cancer. Also, the patient can be a male who has prostate cancer and who has one or more family members who have or have had prostate cancer.

Moreover, in some embodiments, the patient is a female who has not given birth to children. In other embodiments, the patient is a female who has not breastfed children. In yet other embodiments, the patient is a female who has been administered hormones to address the symptoms of menopause for a certain period of time. The patient may have been administered such hormone for more than about 10 years. Furthermore, in some embodiments, the patient is a female who has not taken birth control pills for a certain period of time, such as not taken the pills for about 5 years or more. Moreover, the patient can be a male or female who has been infected by the human papilloma virus (HPV) or genital warts.

In addition, in certain embodiments, patients treated with the methods and compositions described herein may be of certain age range. For instance, the patient can be from about 30 years to about 70 years old, about 30 years to about 50 years old, about 40 years to about 70 years old, or about 65 years to about 85 years old. In some embodiments, the patient is about 30 years old or older, about 35 years old or older, about 40 years old or older, about 45 years old or older, about 50 years old or older, about 55 years old or older, about 60 years old or older, about 65 years old or older, about 70 years old or older, about 75 years old or older, about 80 years old or older, or about 85 years old or older.

In addition, in certain embodiments, the patient is overweight or obese. Also, the patient can have a form of diabetes. The patient can also be a smoker or one who consumes alcohol.

Moreover, in some embodiments, the patient can be of a certain race or ethnicity. For example, the patient may be of Asian-descent, African-descent, European-descent, North American-descent, South American-descent. In some embodiments, the patient may be an African-American male.

The methods and compositions described herein can be used for patients who have or have not previously had cancer. For instance, the patient can be a female who currently has ovarian cancer and who has or had breast cancer. Also, the patient can be one who has or has not been previously treated for cancer. Such patients may have had surgery to treat the cancer or another form of cancer, or may not have had surgery to treat the cancer or another form of cancer. For instance, the patient may have had surgery to remove all or a portion of a cancerous or non-cancerous tumor. Also, the patient may have had surgery to remove all or a portion of a body part or an organ with a cancerous growth, such as a prostate gland, testicle, breast, ovary or uterus. Prostate cancer patients treated can also include those who have been castrated prior to administration of a therapeutically effective amount of a 17α-hydroxylase/C_{17,20}-lyase inhibitor, as well as those who have not been castrated. In other embodiments, the patient may be castration-refractory.

In some embodiments, the patient is chemotherapy-naïve or has not been treated with a particular chemotherapy, such as any of the chemotherapies described herein. In other embodiments, the patient has or is current receiving or will receive chemotherapy. Such chemotherapy includes without limitation the administration of paclitaxel, docetaxel, mitoxantrone, estramustine, satraplatin, and radioisotopes. In certain embodiments, the patient is chemotherapy refractory to a certain chemotherapy, including any of those mentioned herein.

Additionally, in some embodiments, the patient is hormone therapy-naïve or has not been treated with a particular hormone therapy, such as any of the hormone therapies described herein. In other embodiments, the patient has or is current receiving or will receive hormone therapy. Such hormone therapy includes without limitation the administration of hormone ablation agents, hormone deprivation treatment, antiandrogens, ketoconazole, aromatase inhibitors such as aminoglutethimide, flutamide, cyproterone acetate, goserelin, leuprorelin, GnRH agonist, DES, LHRH analogues or steroids, including but not limited to prednisone, hydrocortisone, and dexamethasone. In certain embodiments, the patient is refractory to a certain hormone therapy, including any of those mentioned herein.

With respect to patients that have been previously treated for prostate cancer, in some embodiments the prostate cancers may include metastatic disease, and in other embodiments, the prostate cancers may include non-metastatic disease.

In some embodiments, the prostate cancer patients treated with the methods and compositions described herein include those who have a baseline plasma concentration of prostate-specific antigen (PSA) of about 0.5 ng/mL or more prior to administration of a therapeutically effective amount of a 17α-hydroxylase/C_{17,20}-lyase inhibitor. In other embodiments, the prostate cancer patients treated include those who have a baseline plasma concentration of PSA of about 4.0 ng/mL or more prior to administration of a therapeutically effective amount of a 17α-hydroxylase/C_{17,20}-lyase inhibitor. In certain embodiments, cancer patients treated with the methods and compositions described herein include those who have a baseline plasma concentration of testosterone of about 0.001 ng/dl or more prior to administration of a therapeutically effective amount of a 17α-hydroxylase/C_{17,20}-lyase inhibitor. In other embodiments, the cancer patients treated include those who have a baseline plasma concentration of testosterone of about 250 ng/dl or more prior to administration of a therapeutically effective amount of a 17α-hydroxylase/C_{17,20}-lyase inhibitor.

Moreover, in some embodiments, cancer patients treated with the methods and compositions described herein include those who have a detectable baseline plasma concentration of circulating tumor cells (CTC) prior to administration of a therapeutically effective amount of a 17α-hydroxylase/C_{17,20}-lyase inhibitor. In other embodiments, cancer patients treated include those who have 1 or more CTC per 7.5 mL of blood, or in some instances 4 or more CTCs per 7.5 mL of blood, prior to administration of a therapeutically effective amount of a 17α-hydroxylase/C_{17,20}-lyase inhibitor. In other embodiments, cancer patients treated with the methods and compositions described herein include those who have a tumor having a tumor size detectable on a CT scan prior to administration of a therapeutically effective amount of a 17α-hydroxylase/C_{17,20}-lyase inhibitor. In yet other embodiments, the cancer in the patients has metastasized to for example, the bone, lung, lymph nodes prior to administration of a therapeutically effective amount of a 17α-hydroxylase/C_{17,20}-lyase inhibitor. Furthermore, in some embodiments, cancer patients treated with the methods and compositions described herein include those who have a baseline plasma concentration of alkaline phosphatase (ALP) of about 0.5 IU/L or more prior to administration of a therapeutically effective amount of a 17α-hydroxylase/C_{17,20}-lyase inhibitor. In other embodiments, the cancer patients treated include those who have a baseline plasma concentration of ALP of about 10 IU/L or more prior to administration of a therapeutically effective amount of a 17α-hydroxylase/C_{17,20}-lyase inhibitor. In other embodiments, the cancer patient suffers from pain and is administered a certain amount of a pain relieving agent prior to administration of a therapeutically effective amount of a 17α-hydroxylase/C_{17,20}-lyase inhibitor. Such pain-relieving agents include, but are not limited to, fentanyl, morphine, and OxyContin®.

Determining the Effect of 17α-hydroxylase/C_{17,20}-lyase Inhibitors In Vitro

The methods described herein may be used to inhibit tumor cell proliferation, cell transformation and/or tumorigenesis in vitro and in vivo using a variety of assays known in the art, or described herein. Such activity can be demonstrated in an in vitro assay by contacting a 17α-hydroxylase/C_{17,20}-lyase inhibitor with a tumor cell. In general, a tumor cell is exposed to varying concentrations of a 17α-hydroxylase/C_{17,20}-lyase inhibitor, followed by measuring cell survival rate relative to a control. Such assays can use cells of a cancer cell line or cells from a patient. Many assays well-known in the art can be used to assess such survival and/or growth. For example, cell proliferation may be assayed by measuring (³H)-thymidine incorporation, by direct cell count, by detecting changes in transcription, translation or activity of known genes such as proto-oncogenes (e.g., fos, myc) or cell cycle markers (Rb, dcd2, cyclin A, D1, D2, D3, E, etc.). The levels of such protein and mRNA activity can be determined by any method well known in the art. For example, protein can be quantitated by known immunodiagnostic methods such as Western blotting or immunoprecipitation using commercially available antibodies. mRNA can be quantitated by methods well known and routine in the art, e.g., using Northern analysis, RNase protection or the polymerase chain reaction (PCR) in connection with reverse transcription. Cell viability can be assessed by using trypan-blue staining or other cell death or viability markers known in the art. Differentiation can be assessed, for example, visually based on changes in morphology.

As one example, bromodeoxyuridine (BRDU) incorporation can be used as an in vitro assay to identify proliferating cells. The BRDU assay identifies a cell population undergoing DNA synthesis by incorporation of BRDU into newly synthesized DNA. Newly synthesized DNA can then be detected using an anti-BRDU antibody (see Hoshino et al., Int. J. Cancer 38:369 (1986); Campana et al., J. Immunol. Meth. 107:79 (1988)).

Cell proliferation can also be examined using (³H)-thymidine incorporation (see e.g., Chen, J., Oncogene 13:1395-403 (1996); Jeoung, J., J. Biol. Chem. 270:18367-73 (1995)). This assay allows for quantitative characterization of S-phase DNA synthesis. In this assay, cells synthesizing DNA will incorporate (³H)-thymidine into newly synthesized DNA. Incorporation can then be measured by standard techniques in the art such as by counting of radioisotope in a Scintillation counter (e.g. Beckman LS 3800 Liquid Scintillation Counter).

Detection of proliferating cell nuclear antigen (PCNA) can also be used to measure cell proliferation. PCNA is a 36 kilodalton protein whose expression is elevated in proliferating cells, particularly in early G1 and S phases of the cell cycle and therefore can serve as a marker for proliferating cells. Positive cells are identified by immunostaining using an anti-PCNA antibody (see Li et al., Curr. Biol. 6:189-199 (1996); Vassilev et al., J. Cell Sci. 108:1205-15 (1995)).

Cell proliferation can be measured by counting samples of a cell population over time (e.g. daily cell counts). Cells can be counted using a hemacytometer and light microscopy (e.g. HyLite hemacytometer, Hausser Scientific). Cell number can be plotted against time in order to obtain a growth curve for the population of interest. In a preferred embodiment, cells counted by this method are first mixed with the dye Trypan-blue (Sigma), such that living cells exclude the dye, and are counted as viable members of the population.

DNA content and/or mitotic index of the cells can be measured, for example, based on the DNA ploidy value of the cell. For example, cells in the G1 phase of the cell cycle generally contain a 2N DNA ploidy value. Cells in which DNA has been replicated but have not progressed through mitosis (e.g. cells in S-phase) will exhibit a ploidy value higher than 2N up to 4N DNA content. Ploidy value and cell-cycle kinetics can be further measured using propidum iodide assay (see e.g. Turner, T., et al., Prostate 34:175-81 (1998)). Alternatively, the DNA ploidy can be determined by quantitation of DNA Feulgen staining (which binds to DNA in a stoichiometric manner) on a computerized microdensitometrystaining system (see e.g., Bacus, S., Am. J Pathol. 135:783-92 (1989)). In an another embodiment, DNA content can be analyzed by preparation of a chromosomal spread (Zabalou, S., Hereditas 120:127-40 (1994); Pardue, Meth. Cell Biol. 44:333-351 (1994)).

The expression of cell-cycle proteins (e.g., CycA. CycB, CycE, CycD, cdc2, Cdk4/6, Rb, p21, p27, etc.) provide crucial information relating to the proliferative state of a cell or population of cells. For example, identification in an anti-proliferation signaling pathway can be indicated by the induction of p21^{cip1}. Increased levels of p21 expression in cells results in delayed entry into G1 of the cell cycle (Harper et al., Cell 75:805-816 (1993); Li et al., Curr. Biol. 6:189-199 (1996)). p21 induction can be identified by immunostaining using a specific anti-p21 antibody available commercially (e.g. Santa Cruz). Similarly, cell-cycle proteins can be examined by Western blot analysis using commercially available antibodies. Cell populations may be synchronized prior to detection of a cell cycle protein. Cell cycle proteins can also be detected by FACS (fluorescence-activated cell sorter) analysis using antibodies against the protein of interest.

Detection of changes in length of the cell-cycle or speed of cell-cycle can also be used to measure inhibition of cell proliferation by the 17α-hydroxylase/C_{17,20}-lyase inhibitors. For example, the length of the cell-cycle may be determined by the doubling time of a population of cells (e.g., using cells contacted or not contacted with one or more 17α-hydroxylase/C_{17,20}-lyase inhibitors). Alternatively, FACS analysis may be used to analyze the phase of cell-cycle progression, or purify G1, S, and G2/M fractions (see e.g., Delia, D. et al., Oncogene 14:2137-47 (1997)).

Lapse of cell-cycle checkpoint(s), and/or induction of cell-cycle checkpoint(s), can be examined by the methods described herein, or by any method known in the art. Without limitation, a cell-cycle checkpoint is a mechanism which ensures that a certain cellular events occur in a particular order. Checkpoint genes are defined by mutations that allow late events to occur without prior completion of an early event (Weinert, T., and Hartwell, L., Genetics, 134:63-80 (1993)). Induction or inhibition of cell-cycle checkpoint genes can be assayed, for example, by Western blot analysis, or by immunostaining, etc. Lapse of cell-cycle checkpoints can be further assessed by the progression of a cell through the checkpoint without prior occurrence of specific events (e.g., progression into mitosis without complete replication of the genomic DNA).

In addition to the effects of expression of a particular cell-cycle protein, activity and post-translational modifications of proteins involved in the cell-cycle can play an integral role in the regulation and proliferative state of a cell. Assays involving the detection of post-translational modifications (e.g. phosphorylation) may be conducted by any method known in the art. For example, antibodies that detect phosphorylated tyrosine residues are commercially available, and can be used in Western blot analysis to detect proteins with such modifications. In another example, modifications such as myristylation, can be detected on thin layer chromatography or reverse phase h.p.l.c. (see e.g., Glover, C., Biochem. J. 250:485-91 (1988); Paige, L., Biochem J. 250:485-91 (1988)).

Activity of signaling and cell cycle proteins and/or protein complexes is often mediated by a kinase activity. Analysis of kinase activity may be performed by assays such as the histone H1 assay (see e.g., Delia, D. et al., Oncogene 14:2137-47 (1997)).

The 17α-hydroxylase/C_{17,20}-lyase inhibitor can also be demonstrated to inhibit cell transformation (or progression to malignant phenotype) in vitro. Cells with a transformed cell phenotype may be contacted with one or more 17α-hydroxylase/C_{17,20}-lyase inhibitors, and examined for change in characteristics associated with a transformed phenotype (a set of in vitro characteristics associated with a tumorigenic ability in vivo), for example, but not limited to, colony formation in soft agar, a more rounded cell morphology, looser substratum attachment, loss of contact inhibition, loss of anchorage dependence, release of proteases such as plasminogen activator, increased sugar transport, decreased serum requirement, or expression of fetal antigens, etc. (see Luria et al., 1978, General Virology, 3d Ed., John Wiley & Sons, New York, pp. 436-446).

Loss of invasiveness or decreased adhesion can also be used to demonstrate the anti-cancer effects of the 17α-hydroxylase/C_{17,20}-lyase inhibitor. For example, a critical aspect of the formation of a metastatic cancer is the ability of a precancerous or cancerous cell to detach from primary site of disease and establish a novel colony of growth at a secondary site. The ability of a cell to invade peripheral sites is reflective of a potential for a cancerous state. Loss of invasiveness can be measured by a variety of techniques known in the art including, for example, induction of E-cadherin-mediated cell-cell adhesion. Such E-cadherin-mediated adhesion can result in phenotypic reversion and loss of invasiveness (Hordijk et al., Science 278:1464-66 (1997)).

Loss of invasiveness can be further examined by inhibition of cell migration. A variety of 2-dimensional and 3-dimensional cellular matrices are commercially available (Calbiochem-Novabiochem Corp. San Diego, Calif.). Cell migration across or into a matrix can be examined by microscopy, time-lapsed photography or videography, or by any method in the art allowing measurement of cellular migration. Loss of invasiveness may be examined by response to hepatocyte growth factor (HGF). HGF-induced cell scattering is correlated with invasiveness of cells such as Madin-Darby canine kidney (MDCK) cells. This assay identifies a cell population that has lost cell scattering activity in response to HGF (Hordijk et al., Science 278:1464-66 (1997)).

Alternatively, loss of invasiveness can be measured by cell migration through a chemotaxis chamber (Neuroprobe/Precision Biochemicals Inc. Vancouver, BC). In such assay, a chemo-attractant agent is incubated on one side of the chamber (e.g., the bottom chamber) and cells are plated on a filter separating the opposite side (e.g., the top chamber). In order for cells to pass from the top chamber to the bottom chamber, the cells must actively migrate through small pores in the filter. Checkerboard analysis of the number of cells that have migrated can then be correlated with invasiveness (see e.g., Ohnishi, T., Biochem. Biophys. Res. Commun. 193:518-25 (1993)).

Determining the Effect of 17α-hydroxylase/C_{17,20}-lyase Inhibitors In Vivo

17α-hydroxylase/C_{17,20}-lyase inhibitors can also be demonstrated to inhibit tumor formation in vivo. A wide variety of animal models of hyperproliferative disorders, including tumorigenesis and metastatic spread, are known in the art (see Table 317-1, Chapter 317, “Principles of Neoplasia,” in Harrison's Principals of Internal Medicine, 13^{th} Edition, Isselbacher et al., eds., McGraw-Hill, New York, p. 1814, and Lovejoy et al., 1997, J. Pathol. 181:130-135). General animal models applicable to many types of cancer have been described including, but not limited to, the p53-deficient mouse model (Donehower, 1996, Semin. Cancer Biol. 7:269-278), the Min mouse (Shoemaker et al., Biochem. Biophys. Acta, 1332: F25-F48 (1997)), and immune responses to tumors in rat (Frey, Methods, 12:173-188 (1997)).

For example, a 17α-hydroxylase/C_{17,20}-lyase inhibitor can be administered to a test animal, preferably a test animal predisposed to develop a tumor, and the test animal subsequently examined for a decreased incidence of tumor formation in comparison with controls not administered the 17α-hydroxylase/C_{17,20}-lyase inhibitor. Alternatively, a 17α-hydroxylase/C_{17,20}-lyase inhibitor can be administered to test animals having a tumor (e.g., test animals in which a tumor has been induced by introduction of malignant, neoplastic, or transformed cells, or by administration of a carcinogen) and subsequently examining the tumor in the test animals for tumor regression in comparison to control animals not administered the 17α-hydroxylase/C_{17,20}-lyase inhibitor.

Administration of a 17α-hydroxylase/C_{17,20}-lyase inhibitor, in particular abiraterone acetate, can affect distinct predictive markers of cancers such as prostate cancer, as shown by various in vivo assays. For example, in the prostate cancer arena, the concentration of prostate-specific antigens (PSA) in the blood, as determined by the PSA test, is used to detect and monitor the disease. Although the use of the PSA endpoint has not been validated in a Phase III trial as a surrogate for response or survival, decline in PSA by at least 50% confirmed by a second PSA value four or more weeks later in the absence of clinical or radiographic evidence of disease progression during this time has become a widely used method to screen for activity of a clinical agent in Phase II trials (Pienta, et al., CA Cancer J Clin. 2005; 55:300-318).

Accordingly, administration of a 17α-hydroxylase/C_{17,20}-lyase inhibitor such as abiraterone acetate can cause plasma concentration of PSA in a mammal to decrease to a level which is lower than or less than the plasma concentration of PSA prior to administration. In some embodiments, the 17α-hydroxylase/C_{17,20}-lyase inhibitor is administered in a therapeutically effective amount of about 800 mg/day or more, or in a therapeutically effective amount of about 1000 mg/day or more. Also, in some embodiments, the plasma concentration of PSA post-administration is about 90% or less of the plasma concentration prior to administration, about 75% or less of the plasma concentration prior to administration, or about 50% or less of the plasma concentration prior to administration. In some embodiments, the plasma concentration of PSA post-administration is about 50 ng/mL or less. In other embodiments, the plasma concentration of PSA post-administration is below about 20 ng/mL or less. In yet other embodiments, the plasma concentration of PSA post-administration is below about 10 ng/mL or less. In some embodiments, the plasma concentration of PSA post-administration is less than the plasma concentration of PSA pre-administration for a time period of at least 10 days. The plasma concentration of PSA post-administration may be at or below castration levels of PSA in some embodiments. In these embodiments, the plasma concentration of PSA post-administration is at or below castration level for a time period of at least 10 days.

In addition, plasma concentration of testosterone is another important marker for cancer, such as prostate cancer, as the metabolite of testosterone (dihydrotestosterone (DHT) binds to androgen receptors in a cell and translocates into the cell nucleus where it binds to the androgen-response elements within the DNA, thereby activating the genes involved in cell growth (see id.) Administration of a 17α-hydroxylase/C_{17,20}-lyase inhibitor such as abiraterone acetate can cause plasma concentration of testosterone in a mammal to decrease to a level which is lower than or less than the plasma concentration of testosterone prior to administration. In some embodiments, the 17α-hydroxylase/C_{17,20}-lyase inhibitor is administered in a therapeutically effective amount of about 800 mg/day or more, or in a therapeutically effective amount of about 1000 mg/day or more. Also, in some embodiments, the plasma concentration of testosterone post-administration is about 75% or less of the plasma concentration prior to administration, or about 50% or less of the plasma concentration prior to administration. In some embodiments, the plasma concentration of testosterone post-administration is less than the plasma concentration of testosterone pre-administration for a time period of at least 10 days. In some embodiments, the plasma concentration of testosterone post-administration is about 250 ng/dl or less. In other embodiments, the plasma concentration of testosterone post-administration is below about 0.001 ng/dl or less. In other embodiments, the plasma concentration of testosterone post-administration is at or below castration levels of testosterone. In some embodiments, the plasma concentration of testosterone post-administration is at or below castration levels of testosterone for a time period of at least 10 days.

Another marker for monitoring cancer is the number of circulating tumor cells (CTC) found in the patient's body. Administration of a 17α-hydroxylase/C_{17,20}-lyase inhibitor such as abiraterone acetate can cause the number of CTCs in a mammal to decrease to a number which is smaller or fewer than the number of CTCs prior to administration. In some embodiments, the 17α-hydroxylase/C_{17,20}-lyase inhibitor is administered in a therapeutically effective amount of about 800 mg/day or more, or in a therapeutically effective amount of about 1000 mg/day or more. Also, in some embodiments, the number of CTCs post-administration is about 75% or less of the number of CTCs prior to administration, or about 50% or less of the number of CTCs prior to administration. In some embodiments, the number of CTC post-administration is about 100 or less per 7.5 ml of blood, about 50 or less per 7.5 ml of blood, 25 or less per 7.5 ml of blood, or about 4 or less per 7.5 ml of blood. In other embodiments, the number of CTC post-administration is not detectable.

An additional marker for monitoring cancer in a patient is tumor size or tumor mass. Administration of a 17α-hydroxylase/C_{17,20}-lyase inhibitor such as abiraterone acetate can cause a tumor in a mammal to stop growing or can cause the size or mass of a tumor in a mammal to decrease compared to the tumor size or mass prior to administration. In some embodiments, the 17α-hydroxylase/C_{17,20}-lyase inhibitor is administered in a therapeutically effective amount of about 800 mg/day or more, or in a therapeutically effective amount of about 1000 mg/day or more. In addition in some embodiments, the tumor mass post-administration is about 10% or less of the tumor mass prior to administration, about 25% or less of the tumor mass prior to administration or about 50% or less of the tumor mass prior to administration. The above tumor mass reductions may be found in about 30% or more of patients treated with the methods and compositions described herein.

An additional marker for monitoring cancer is the extent of bone metastasis or lymph node metastasis. Administration of a 17α-hydroxylase/C_{17,20}-lyase inhibitor such as abiraterone acetate to a mammal wherein the cancer has metastasized to a bone or lymph node before administration can result in reduction or stabilization of bone or lymph node metastasis in the mammal post-administration. In some embodiments, the 17α-hydroxylase/C_{17,20}-lyase inhibitor is administered in a therapeutically effective amount of about 800 mg/day or more, or in a therapeutically effective amount of about 1000 mg/day or more. In certain embodiments, the size of the metastasis is reduced by 10% or more post-administration, or 30% or more post-administration.

Another marker for monitoring cancer is plasma concentration of alkaline phosphatase (ALP). Administration of a 17α-hydroxylase/C_{17,20}-lyase inhibitor such as abiraterone acetate can result in plasma concentration of ALP in a mammal decreasing to a level which is lower than or less than the plasma concentration of ALP prior to administration. In some embodiments, the 17α-hydroxylase/C_{17,20}-lyase inhibitor is administered in a therapeutically effective amount of about 800 mg/day or more, or in a therapeutically effective amount of about 1000 mg/day or more. Also, in some embodiments, the plasma concentration of ALP post-administration is about 10% or less of the plasma concentration of ALP prior to administration, or about 30% or less of the plasma concentration of ALP prior to administration. In other embodiments, the plasma concentration of ALP post-administration is about 50 IU/L or less, about 25 IU/L or less, about 10 IU/L or less, about 1 IU/L or less, or about 0.5 IU/L or less.

Furthermore, the amount of pain experienced by a patient is another marker for the extent of a cancerous disease. Administration of a 17α-hydroxylase/C_{17,20}-lyase inhibitor such as abiraterone acetate can result in a decreased level of pain experienced by a patient post-administration when compared to the level of pain experienced by the patient prior to administration. In some embodiments, the 17α-hydroxylase/C_{17,20}-lyase inhibitor is administered in a therapeutically effective amount of about 800 mg/day or more, or in a therapeutically effective amount of about 1000 mg/day or more.

Moreover, the amount of a pain-relieving agent or agents administered before and after administration of a clinical agent may be indicative of response to that clinical agent. Administration of a 17α-hydroxylase/C_{17,20}-lyase inhibitor such as abiraterone acetate can result in a reduced need for pain-relieving agents, which means that the amount of the pain-relieving agent given to the patient post-administration will be less than the amount given pre-administration. In some embodiments, the 17α-hydroxylase/C_{17,20}-lyase inhibitor is administered in a therapeutically effective amount of about 800 mg/day or more, or in a therapeutically effective amount of about 1000 mg/day or more. Moreover, in some embodiments, the amount of the pain-relieving agent given post-administration is about 30% or less of the amount given pre-administration, or about 10% or less of the amount given pre-administration.

The description contained herein is for purposes of illustration and not for purposes of limitation. The methods and compositions described herein can comprise any feature described herein either alone or in combination with any other feature(s) described herein. Changes and modifications may be made to the embodiments of the description. Furthermore, obvious changes, modifications or variations will occur to those skilled in the art. Also, all references cited above are incorporated herein, in their entirety, for all purposes related to this disclosure.

The following illustrative examples are set forth to assist in understanding the methods and compositions described herein and do not limit the claimed methods and compositions.

### EXAMPLE

An open label phase I/II study was carried out to evaluate the safety, efficacy and recommended dose of abiraterone acetate in prostate cancer patients. Pharmacokinetics (PK), pharmacodynamics and antitumor efficacy were also studied.

### Part A: Pharmacokinetic Study of Abiraterone in Hormone Refractory Prostate Cancer Patients

A group of 15 subjects was divided into five groups. Members of each group were orally administered doses of 250 mg/day, 500 mg/day, 750 mg/day, 1000 mg/day or 2000 mg/day once a day. Before administration the subjects had fasted. PK blood samples were measured at 1, 2, 4, 6, 8, 24, and 72 hours post dose.

To measure the amount of abiraterone and abiraterone acetate in the samples, these compounds were extracted from 100 uL human plasma using solid phase extraction (SPE) with cation mixed-mode polymer cartridges. Extracts were evaporated to dryness before reconstitution and analysis. Chromatography was carried out using a Luna™ C5 column (5 μm, 50 mm×2.1 mm id) with a gradient mobile phase consisting of 10 mM ammonium acetate and 2% propan-2-ol in acetonitrile. The flow rate was 0.2 mL/min and the run time 10.5 minutes. Abiraterone and abiraterone acetate were ionized using the electrospray interface in positive ion mode. Detection of analytes was via tandem mass spectrometry (MS/MS) in the multiple reaction monitoring (MRM) mode.

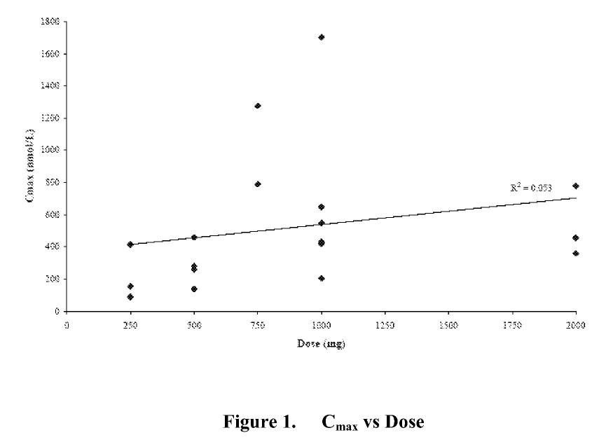

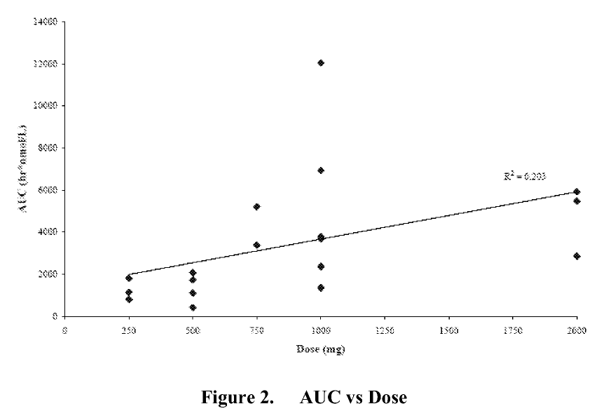

Table 1 below shows the mean PK parameters for abiraterone in the blood samples of the subjects. FIG. 1 shows a plot of the maximum measured plasma concentration of abiraterone as a function of the doses tested. FIG. 2 shows a plot of the area under the curve (AUC) as a function of the doses tested.

PK modeling was conducted using a non-compartmental model, extra vascular input for plasma with WinNonLin Software®. Pharmacokinetic parameters estimated in the model include:

| | | |
|---|---|---|
| Cₘₐₓ | (nmol/L) | Maximum concentration |
| | | corresponding to Tₘₐₓ |
| T_{max:} | (hr): | Time of maximum observed |
| | | concentration |
| HL_Lambda_z | (hr): | Terminal half-life of the drug |
| Cl_F_obs | (L/h): | Total body clearance |
| Vz_F_obs | (L): | Volume of distribution |
| AUClast | (hr * nmol/L): | Area under concentration v time |
| | | curve from the time of dosing to the |
| | | last measurable concentration |
| AUCINF_obs | (hr * nmol/L): | Area under concentration v time |
| | | curve from the time of dosing |
| | | extrapolated to infinity |

TABLE 1
Mean pharmacokinetic parameters for Abiraterone (fasted)

| | | Tmax | Cmax | HL_Lambda_z | AUClast | AUCINF_obs | Cl_F_obs | Vz_F_obs |
|---|---|---|---|---|---|---|---|---|
| Dose | | (hr) | (nmol/L) | (hr) | (hr * nmol/L) | (hr * nmol/L) | (L/hr) | (L) |
| 250 | Mean | 2.05 | 219 | 11.6 | 1253 | 1369 | 567.2 | 10772.0 |
| | SD | 0.02 | 172.77 | 10.14 | 519.78 | 505.39 | 183.18 | 11902.44 |
| | % CV | 0.83 | 78.77 | 87.05 | 41.48 | 36.93 | 32.29 | 110.49 |
| 500 | Mean | 2.588 | 284 | 9.5 | 1334 | 1448 | 1347.2 | 14032.5 |
| | SD | 1.02 | 132.38 | 7.02 | 731.47 | 749.01 | 991.12 | 5660.2 |
| | % CV | 39.25 | 46.62 | 73.74 | 54.83 | 51.74 | 73.57 | 40.34 |
| 750 | Mean | 1.709 | 1032 | 10.8 | 4294 | 4537 | 494 | 8340 |
| | SD | 0/41 | 344.01 | 5.91 | 1295.2 | 1333.42 | 145.27 | 6481.8 |
| | % CV | 24.13 | 33.33 | 54.57 | 30.17 | 29.39 | 29.39 | 77.72 |
| 1000 | Mean | 3.086 | 659 | 9.9 | 5034 | 5316.4 | 861.6 | 10125.5 |
| | SD | 1.74 | 531.85 | 3.52 | 3919.47 | 4207.55 | 618.58 | 4698.97 |
| | % CV | 56.49 | 80.69 | 35.62 | 77.86 | 79.14 | 71.79 | 46.41 |
| 2000 | Mean | 2.672 | 531 | 12.0 | 4754 | 4983 | 1280.3 | 21598.8 |
| | SD | 1.14 | 219.02 | 2.29 | 1659.13 | 1755.24 | 549.76 | 7227.17 |
| | % CV | 42.50 | 41.23 | 19.01 | 34.90 | 35.22 | 42.94 | 33.46 |

*Statistics calculated on non-rounded figures

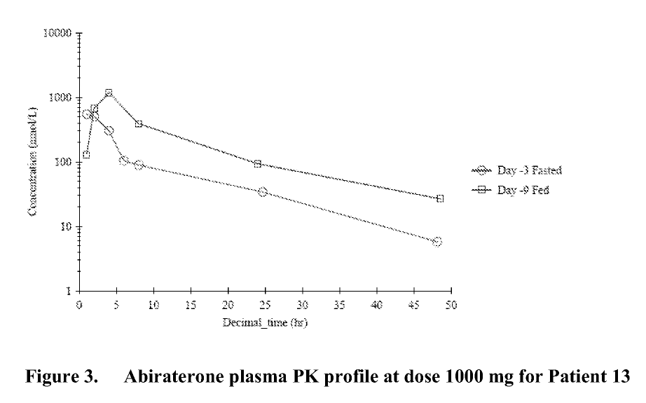

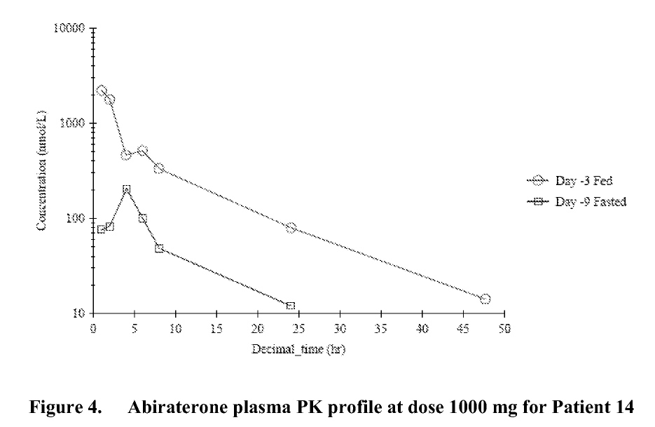

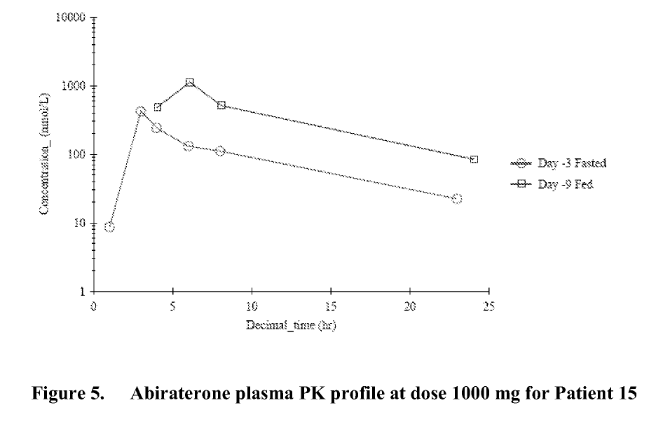

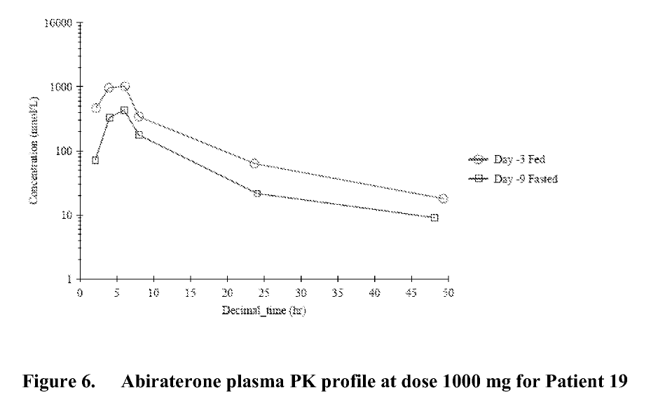

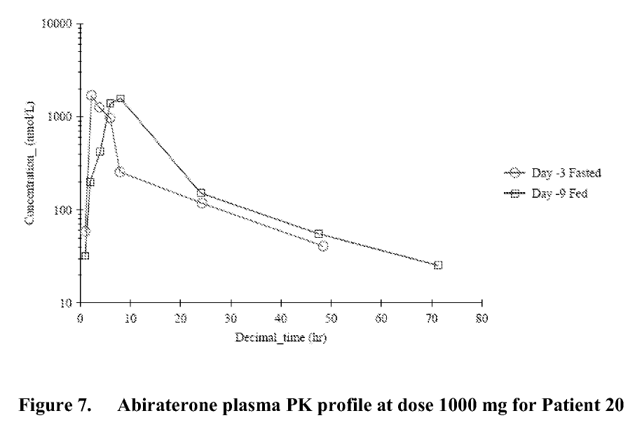

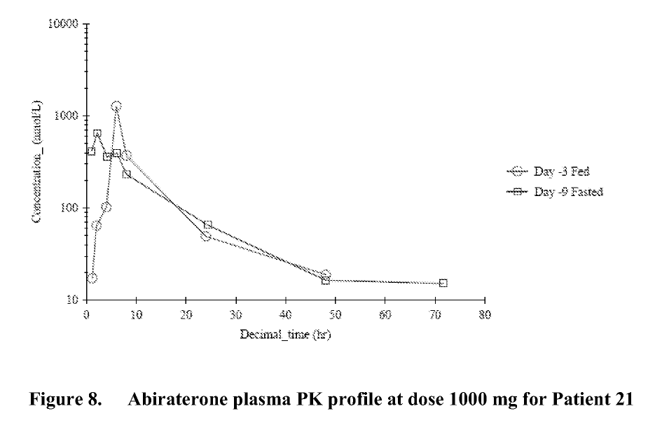

Based on the above results, the PK parameters for a single dose of 1000 mg/day of abiraterone acetate were further studied in the 3 subjects who were administered the 1000 mg/day dose and in 3 additional subjects. Two single oral doses (1000 mg) of abiraterone acetate were administered separately, one concomitantly with food of high fat content and one in fasting state. The first single dose was administered on day −9 with PK blood samples being taken at 1, 2, 4, 6, 8, 24, 48 and 72 hours post dose. The second single dose was administered on day −3 with PK blood samples being taken at 1, 2, 4, 6, 8, 24 and 48 hours post dose for analysis. Tables 2 to 4 and FIGS. 3 to 8 show the results.

TABLE 2
Calculated Abiraterone concentration in plasma (nM) for
Patients 13 and 14

| | | Abiraterone | | | |
|---|---|---|---|---|---|
| | Sample Time | Concentration (nM) | | | Dose |
| Sample | (hr) | Fed | Fasted | Patient | (mg) |
| ABIP169 | pre-dose (day −9) | 0.0 | x | 13 | 1000 |
| ABIP170 | 1.000 | 130.0 | x | 13 | 1000 |
| ABIP171 | 2.000 | 675.0 | x | 13 | 1000 |
| ABIP172 | 4.000 | 1184.5 | x | 13 | 1000 |
| ABIP173 | 6.000 | excluded | x | 13 | 1000 |
| ABIP174 | 7.983 | 392.2 | x | 13 | 1000 |
| ABIP175 | 23.983 | 93.7 | x | 13 | 1000 |
| ABIP176 | 48.500 | 26.8 | x | 13 | 1000 |
| ABIP177 | 72.000 | BLLOQ¹ | x | 13 | 1000 |
| ABIP178 | pre-dose (day −3) | x | 0.0 | 13 | 1000 |
| ABIP179 | 1.033 | x | 548.5 | 13 | 1000 |
| ABIP180 | 2.017 | x | 500.5 | 13 | 1000 |
| ABIP181 | 4.000 | x | 304.2 | 13 | 1000 |
| ABIP182 | 6.000 | x | 104.0 | 13 | 1000 |
| ABIP183 | 8.000 | x | 89.4 | 13 | 1000 |
| ABIP184 | 24.667 | x | 34.1 | 13 | 1000 |
| ABIP185 | 48.117 | x | 5.8 | 13 | 1000 |
| ABIP197 | pre-dose (day −9) | x | 0.0 | 14 | 1000 |
| ABIP198 | 1.000 | x | 76.3 | 14 | 1000 |
| ABIP199 | 2.000 | x | 82.5 | 14 | 1000 |
| ABIP200 | 4.067 | x | 204.7 | 14 | 1000 |
| ABIP201 | 6.050 | x | 99.8 | 14 | 1000 |
| ABIP202 | 8.017 | x | 48.6 | 14 | 1000 |
| ABIP203 | 24.017 | x | 12.1 | 14 | 1000 |
| ABIP204 | 48.217 | x | BLLOQ | 14 | 1000 |
| ABIP205 | 71.333 | x | 0.0 | 14 | 1000 |
| ABIP206 | pre-dose (day −3) | 0.0 | x | 14 | 1000 |
| ABIP207 | 1.033 | 2206.5 | x | 14 | 1000 |
| ABIP208 | 2.033 | 1773.5 | x | 14 | 1000 |
| ABIP209 | 4.000 | 463.5 | x | 14 | 1000 |
| ABIP210 | 6.000 | 516.0 | x | 14 | 1000 |
| ABIP211 | 7.983 | 334.9 | x | 14 | 1000 |
| ABIP212 | 24.083 | 79.7 | x | 14 | 1000 |
| ABIP213 | 47.700 | 14.2 | x | 14 | 1000 |

¹BLLOQ: below lower limit of quantification

TABLE 3
Calculated Abiraterone concentration in plasma (nM) for
Patients 15 and 19

| | | Abiraterone | | | |
|---|---|---|---|---|---|
| | Sample Time | Concentration (nM) | | | Dose |
| Sample | (hr) | Fed | Fasted | Patient | (mg) |
| ABIP225 | pre-dose (day −9) | 0.0 | x | 15 | 1000 |
| ABIP226 | 1.000 | 0.0 | x | 15 | 1000 |
| ABIP227 | 2.000 | BLLOQ | x | 15 | 1000 |
| ABIP228 | 4.033 | 484.0 | x | 15 | 1000 |
| ABIP229 | 6.083 | 1120.5 | x | 15 | 1000 |
| ABIP230 | 8.067 | 511.5 | x | 15 | 1000 |
| ABIP231 | 24.050 | 84.3 | x | 15 | 1000 |
| ABIP232 | 46.050 | 18.1 | x | 15 | 1000 |
| ABIP233 | 70.100 | BLLOQ | x | 15 | 1000 |
| ABIP234 | pre-dose (day −3) | x | BLLOQ | 15 | 1000 |
| ABIP235 | 1.000 | x | 8.7 | 15 | 1000 |
| ABIP236 | 2.983 | x | 419.6 | 15 | 1000 |
| ABIP237 | 3.967 | x | 241.9 | 15 | 1000 |
| ABIP238 | 5.983 | x | 131.2 | 15 | 1000 |
| ABIP239 | 8.000 | x | 110.8 | 15 | 1000 |
| ABIP240 | 22.983 | x | 22.4 | 15 | 1000 |
| ABIP241 | 47.267 | x | excluded | 15 | 1000 |
| ABIP319 | pre-dose (day −9) | x | 0.0 | 19 | 1000 |
| ABIP320 | 1.050 | x | 0.0 | 19 | 1000 |
| ABIP321 | 2.083 | x | 71.9 | 19 | 1000 |
| ABIP322 | 4.017 | x | 333.5 | 19 | 1000 |
| ABIP323 | 6.000 | x | 432.1 | 19 | 1000 |
| ABIP324 | 8.017 | x | 177.0 | 19 | 1000 |
| ABIP325 | 24.067 | x | 21.6 | 19 | 1000 |
| ABIP326 | 48.100 | x | 9.1 | 19 | 1000 |
| ABIP327 | 71.950 | x | BLLOQ | 19 | 1000 |
| ABIP328 | pre-dose (day −3) | 0.0 | x | 19 | 1000 |
| ABIP329 | 0.967 | 0.0 | x | 19 | 1000 |
| ABIP33O | 2.200 | 466.1 | x | 19 | 1000 |
| ABIP331 | 3.933 | 973.0 | x | 19 | 1000 |
| ABIP332 | 6.133 | 1017.0 | x | 19 | 1000 |
| ABIP333 | 8.000 | 344.0 | x | 19 | 1000 |
| ABIP334 | 23.717 | 63.6 | x | 19 | 1000 |
| ABIP335 | 49.300 | 18.0 | x | 19 | 1000 |

TABLE 4
Calculated Abiraterone concentration in plasma (nM) for
Patients 20 and 21

| | | Abiraterone | | | |
|---|---|---|---|---|---|
| | Sample Time | Concentration (nM) | | | Dose |
| Sample | (far) | Fed | Fasted | Patient | (mg) |
| ABIP341 | pre-dose (day −9) | BLLOQ | x | 20 | 1000 |
| ABIP342 | 1.017 | 32.2 | x | 20 | 1000 |
| ABIP343 | 2.000 | 200.7 | x | 20 | 1000 |
| ABIP344 | 4.000 | 422.0 | x | 20 | 1000 |
| ABIP345 | 6.000 | 1396.0 | x | 20 | 1000 |
| ABIP346 | 8.033 | 1582.0 | x | 20 | 1000 |
| ABIP347 | 24.000 | 152.0 | x | 20 | 1000 |
| ABIP348 | 47.517 | 55.4 | x | 20 | 1000 |
| ABIP349 | 71.333 | 25.3 | x | 20 | 1000 |
| ABIP35O | pre-dose (day −3) | x | 5.1 | 20 | 1000 |
| ABIP351 | 1.017 | x | 58.5 | 20 | 1000 |
| ABIP352 | 2.267 | x | 1701.5 | 20 | 1000 |
| ABIP353 | 3.833 | x | 1268.5 | 20 | 1000 |
| ABIP354 | 6.000 | x | 976.0 | 20 | 1000 |
| ABIP355 | 7.917 | x | 256.5 | 20 | 1000 |
| ABIP356 | 24.283 | x | 118.0 | 20 | 1000 |
| ABIP357 | 48.417 | x | 40.8 | 20 | 1000 |
| ABIP363 | pre-dose (day −9) | x | 0 | 21 | 1000 |
| ABIP364 | 1.000 | x | 413.9 | 21 | 1000 |
| ABIP365 | 2.167 | x | 648.5 | 21 | 1000 |
| ABIP366 | 4.150 | x | 359.5 | 21 | 1000 |
| ABIP367 | 6.117 | x | 396.1 | 21 | 1000 |
| ABIP368 | 8.050 | x | 230.9 | 21 | 1000 |
| ABIP369 | 24.333 | x | 65.5 | 21 | 1000 |
| ABIP370 | 48.083 | x | 16.4 | 21 | 1000 |
| ABIP371 | 71.700 | x | 15.2 | 21 | 1000 |
| ABIP372 | pre-dose (day −3) | BLLOQ | x | 21 | 1000 |
| ABIP373 | 1.167 | 17.3 | x | 21 | 1000 |
| ABIP374 | 2.000 | 64.3 | x | 21 | 1000 |
| ABIP375 | 4.000 | 102.2 | x | 21 | 1000 |
| ABIP376 | 6.000 | 1278.5 | x | 21 | 1000 |
| ABIP377 | 8.033 | 371.6 | x | 21 | 1000 |
| ABIP378 | 24.000 | 49.3 | x | 21 | 1000 |
| ABIP379 | 48.083 | 18.9 | x | 21 | 1000 |

The data shown in Tables 2-4 was used to construct concentration versus time plasma PK profiles. The profile for abiraterone is shown in FIGS. 3-8.

TABLE 5
Non-compartmental pharmacokinetic analysis parameters for Abiraterone

| | Dose | Fed/ | Tmax | Cmax | HL | AUClast | AUCINF | Cl F obs | Vz F obs |
|---|---|---|---|---|---|---|---|---|---|
| Patient | (mg) | Fasted | (hr) | (nmol/L) | (hr) | (hr * nmol/L) | (hr * nmol/L) | (L/hr) | (L) |
| Patient 13 | 1000 | Fed | 4.000 | 1184.5 | 10.7 | 10832 | 11244 | 254.5 | 3917.1 |
| | | Fasted | 1.033 | 548.5 | 10.2 | 3696 | 3781 | 756.7 | 11138.0 |
| Patient 14 | 1000 | Fed | 1.033 | 2206.5 | 8.8 | 11599 | 11779 | 242.9 | 3071.1 |
| | | Fasted | 4.067 | 204.7 | 6.6 | 1348 | 1462 | 1956.6 | 18516.0 |
| Patient 15 | 1000 | Fed | 6.083 | 1120.5 | 8.0 | 10127 | 10335 | 276.8 | 3186.5 |
| | | Fasted | 2.983 | 419.6 | 6.6 | 2372 | 2586 | 1106.5 | 10538.1 |
| Patient 19 | 1000 | Fed | 6.133 | 1017.0 | 10.0 | 9466 | 9726 | 294.2 | 4245.3 |
| | | Fasted | 6.000 | 432.1 | 8.0 | 3803 | 3908 | 732.1 | 8488.4 |
| Patient 20 | 1000 | Fed | 8.033 | 1582.0 | 18.3 | 22842 | 23510 | 121.7 | 3213.5 |
| | | Fasted | 2.267 | 1701.5 | 15.3 | 12050 | 12951 | 220.9 | 4878.1 |
| Patient 21 | 1000 | Fed | 6.000 | 1278.5 | 9.7 | 7450 | 7714 | 370.9 | 5179.8 |
| | | Fasted | 2.167 | 648.5 | 12.6 | 6934 | 7210 | 396.8 | 7194.3 |

* Figures rounded up after WinNonLin Analysis

TABLE 6
Mean pharmacokinetic parameters for Abiraterone at 1000 mg

| | | Tmax | Cₘₐₓ | HL | AUClast | AUCINF | Cl F obs | Vz F obs |
|---|---|---|---|---|---|---|---|---|
| | | (hr) | (nmol/L) | (hr) | (hr * nmol/L) | (hr * nmol/L) | (L/hr) | (L) |
| Fed | Mean | 5.214 | 1398 | 10.9 | 12053 | 12385 | 260.2 | 3802.2 |
| | SD | 2.41 | 440.35 | 3.75 | 5471.50 | 5630.70 | 81.48 | 820.67 |
| | % CV | 46.29 | 31.50 | 34.39 | 45.40 | 45.46 | 31.32 | 21.58 |
| Fasted | Mean | 3.086 | 659 | 9.9 | 5034 | 5316 | 861.6 | 10125.5 |
| | SD | 1.74 | 531.85 | 3.52 | 3919.47 | 4207.55 | 618.58 | 4698.97 |
| | % CV | 56.49 | 80.69 | 35.62 | 77.86 | 79.14 | 71.79 | 46.41 |

*Statistics calculated on non-rounded figures
SD standard of deviation
CV coefficient of variation

Following an oral dose of abiraterone acetate at 1000 mg, no abiraterone acetate was detected in vivo. Inter patient variability observed within the cohort was no greater than that observed at other dose levels, i.e. about 6 fold variation in AUC and 3 fold variation in Cmax (Tables 5-6).

Overall, drug levels measured were not higher than previously seen with a dose of 750 mg in a fasting state, where a mean maximum drug concentration (Cmax) of 1032 nM was measured. However, the drug levels at 1000 mg are higher than that observed following 500 mg Abiraterone acetate. Previously the mean drug exposure (AUC) calculated at 2000 mg was approximately double that of 1000 mg in both dosing regimes. However, in the extended cohort there was still an approximate doubling of AUC in the fed group but no longer in the fasted group.

Abiraterone plasma levels were higher when administered concomitantly with food, in comparison with fasting (See Tables 2-6 and FIGS. 3-8). Previously, up to a four fold difference in mean drug levels was observed between the dosing regimes. Up to a two fold difference in mean drug levels was observed in the extended cohort of six patients (Tables 2-6). Out of all the patients enrolled in the food effect study, patient 21 was the only patient where the effect of food was negligible.

### Part B: Evaluation of the Effect of Abiraterone on Certain Prostate Cancer Markers

The effect of abiraterone acetate on the blood levels of prostate-specific antigen (PSA) was evaluated. Two groups of subjects were involved in the study. The first group (“Group 1”) was comprised of the 18 subjects discussed above as well as 12 additional subjects that were administered 1000 mg/day of abiraterone acetate. All 30 of the subjects in Group 1 were castrated and hormone refractory; however, none of the 30 had been treated with chemotherapy, i.e., they were chemotherapy-naïve. The second group (“Group 2”) was comprised of 13 subjects, all of whom were castrated and hormone refractory. Also, these 13 subjects had been administered docetaxel and were chemotherapy refractory. The subjects in Group 2 were administered 1000 mg/day of abiraterone acetate. The subjects in both groups were administered abiraterone acetate until they progressed. No dose limiting toxicity (DLTs) were observed in any of the subjects tested

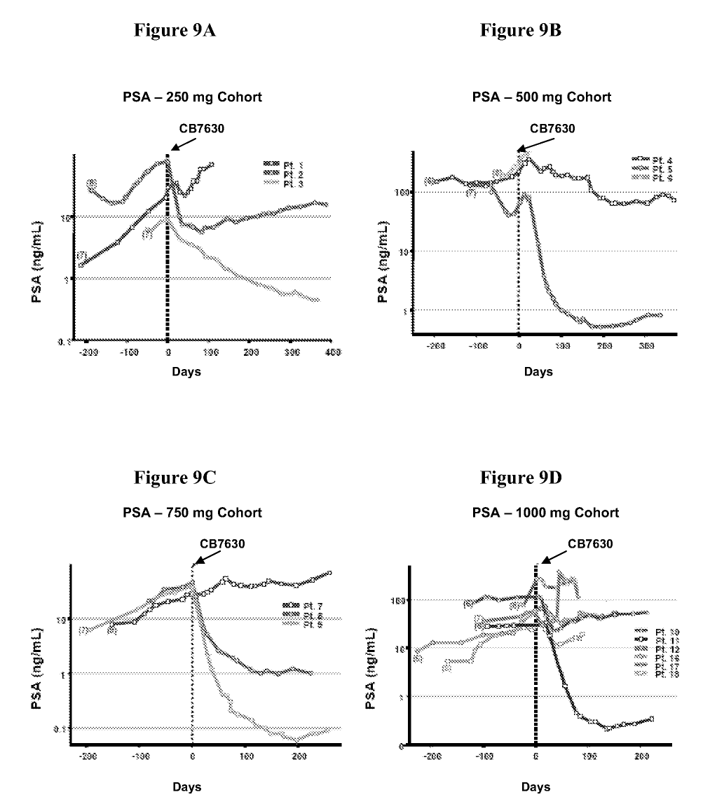

60% of the Group 1 subjects (18 of 30) experienced a decline of PSA levels of greater than 50% for more than 10 days. A third of these subjects experienced PSA declines of greater than 90% for more than 10 days. FIGS. 9A-9D show the effect of the administration of abiraterone acetate on PSA levels for certain of the Group 1 subjects that were administered 250 mg/day, 500 mg/day, 750 mg/day and 1000 mg/day of abiraterone acetate. With respect to the Group 2 subjects, 7 of the 13 subjects experienced a decline in PSA levels of about 50% for more than 10 days. A decrease in analgesic use was observed in the subjects of both groups.

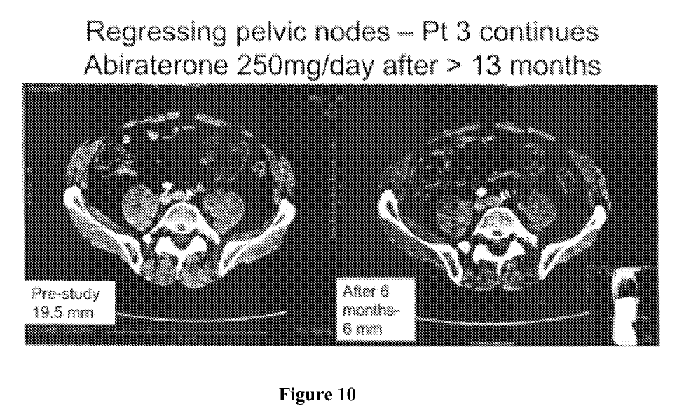

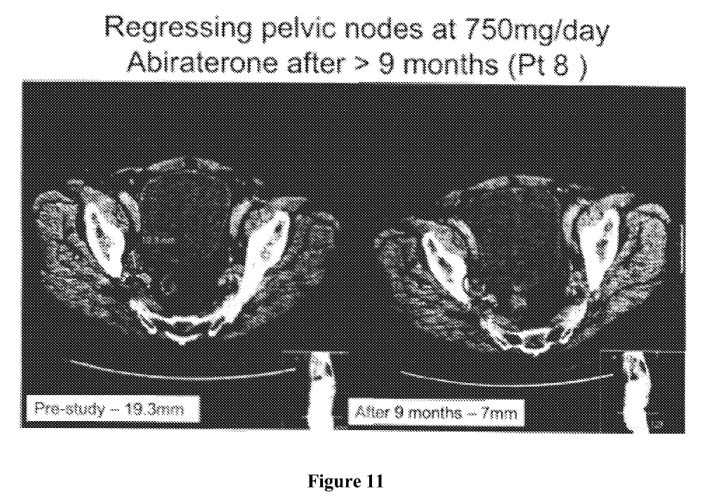

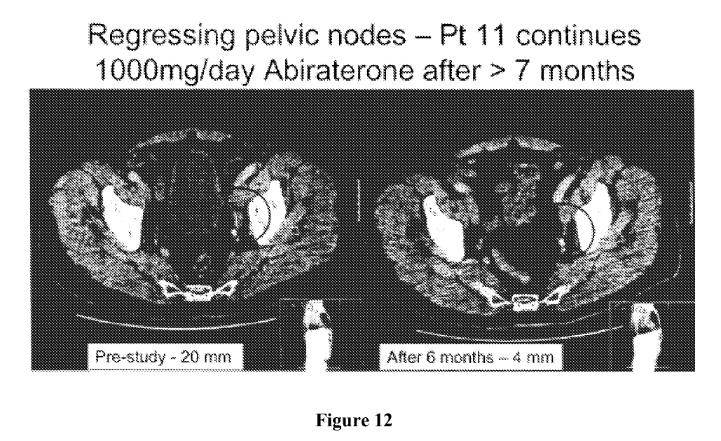

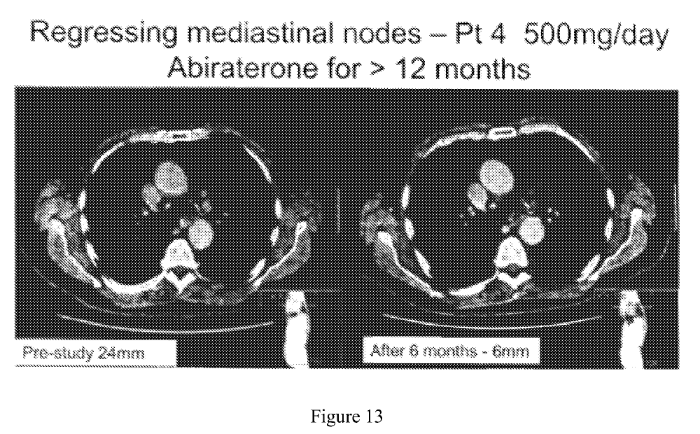

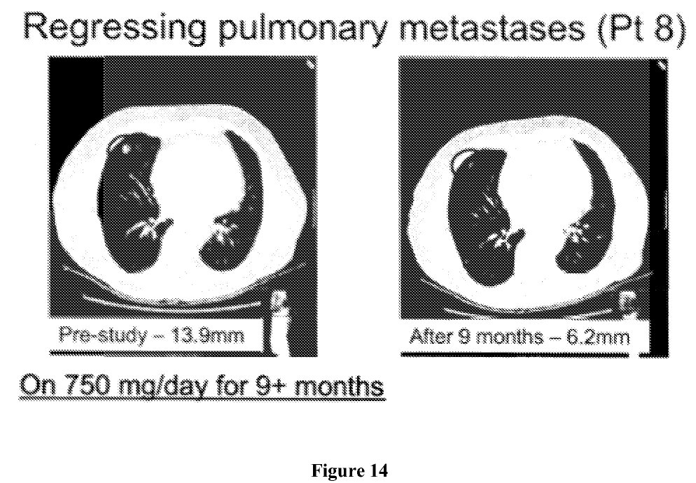

The effect of the administration of abiraterone acetate on tumor mass size was observed. In 20 evaluable Group 1 subjects, who had measurable tumor lesions, administration of abiraterone acetate resulted in partial radiological responses in 11 subjects as measured by CT scans (RECIST). Seven of the subjects demonstrated ongoing stable bone disease and three experienced regressing bone disease. FIGS. 10, 11 and 12 show the regression of pelvic nodes in subjects that were administered 250 mg/day, 750 mg/day and 1000 mg/day of abiraterone acetate, respectively. Also, FIG. 13 shows the regression of mediastinal nodes in a subject that was administered 500 mg/day of abiraterone acetate and FIG. 14 shows the regression of pulmonary metastases for a subject that was administered 750 mg/day of abiraterone acetate.

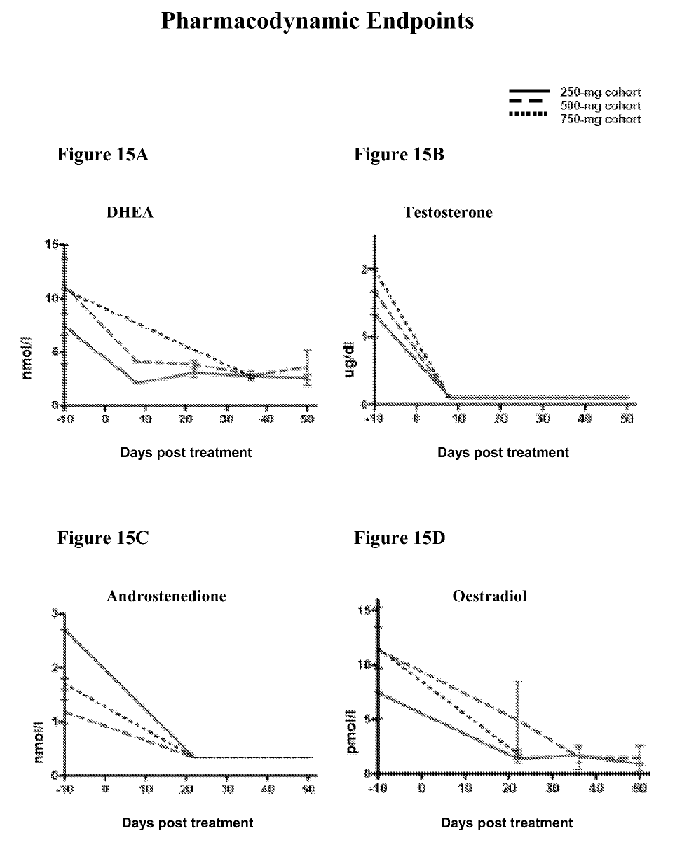

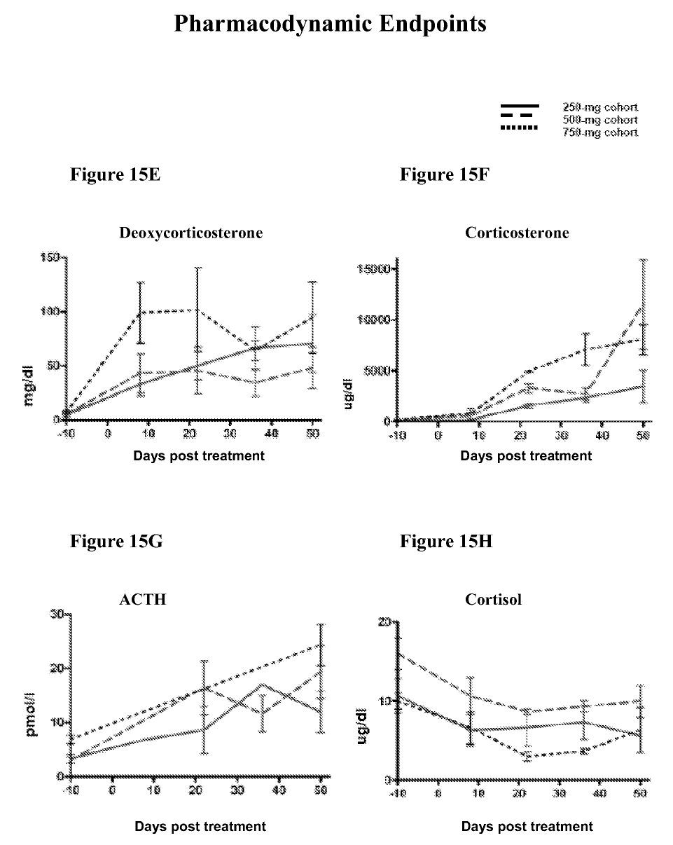

Also, declines in the number of circulating tumor cells (CTC) were observed in certain patients after administration of abiraterone acetate. Furthermore, declines in the blood plasma levels of alkaline phosphatase (ALP) as well as androgenic steroids, such as testosterone, were observed in some subjects. FIGS. 15A-15H show the blood plasma level of certain steroids over time in subjects that were administered 250 mg/day, 500 mg/day and 750 mg/day of abiraterone acetate.

What is claimed is:

1. A method for the treatment of a patient having cancer comprising administering to the patient having cancer that is (A) refractory to a chemotherapy, or (B) refractory to a chemotherapy and a hormone therapy, a therapeutically effective amount of a 17α-hydroxylase/C_{17,20}-lyase inhibitor.

2. The method of claim 1, wherein the 17α-hydroxylase/C_{17,20}-lyase inhibitor comprises a compound of the formula (I) or derivative, analog, pharmaceutically acceptable salt thereof 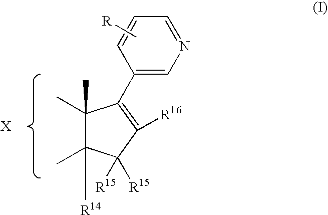
wherein X represents the residue of the A, B and C rings of a steroid which can be, without limitation, androstan-3α- or 3β-ol; androst-5-en-3α- or 3β-ol; androst-4-en-3-one; androst-2-ene; androst-4-ene; androst-5-ene; androsta-5,7-dien-3α or 3β-ol; androsta-1,4-dien-3-one; androsta-3,5-diene; androsta-3,5-diene-3-ol; estra-1,3,5[10]-triene; estra-1,3,5[10]-trien-3-ol; 5α-androstan-3-one; androst-4-ene-3,11-dione; 6-fluoroandrost-4-ene-3-one; or androstan-4-ene-3,6-dione; each of which, where structurally permissible, can be further derivatized in one or more of the following ways, including, but not limited to, to form 3-esters; to have one or more carbon or carbon ring double bonds in any of the 5,6-, 6,7-, 7,8-, 9,11- and 11,12-positions; as 3-oximes; as 3-methylenes; as 3-carboxylates; as 3-nitriles; as 3-nitros; as 3-desoxy derivatives; to have one or more hydroxy, halo, C₁₋₄-alkyl, trifluoro-methyl, C₁₋₄-alkoxy, C₁₋₄-alkanoyloxy, benzoyloxy, oxo, methylene or alkenyl substituents in the A, B, or C-ring; or to be 19-nor;
R represents a hydrogen atom or an alkyl group of 1-4 carbon atoms;
R¹⁴ represents a hydrogen atom, a halogen atom or an alkyl group of 1 to 4 carbon atoms;
each of the R¹⁵ substituents independently represents a hydrogen atom or an alkyl or alkoxy group of 1-4 carbon atoms, a hydroxy group or an alkylcarbonyloxy group of 2 to 5 carbon atoms or together represent an oxo or methylene group or R¹⁴ and one of the R¹⁵ groups together represent a double bond and the other R¹⁵ group represents a hydrogen atom or an alkyl group of 1 to 4 carbon atoms; and
R¹⁶ represents a hydrogen atom, halogen atom, or an alkyl group of 1 to 4 carbon atoms,
in the form of the free bases or pharmaceutically acceptable acid addition salts, but excluding 3β-acetoxy-17-(3-pyridyl)androsta-5,14,16-triene, 3β,15α- and 3β,15β-diacetoxy-17-(3-pyridyl)androsta-5,16-diene and 3β-methoxy-17-(3-pyridyl-5α-androst-16-ene.

3. The method of claim 1, wherein the 17α-hydroxylase/C_{17,20}-lyase inhibitor comprises abiraterone acetate or a pharmaceutically acceptable salt thereof.

4. The method of claim 3, wherein the abiraterone acetate is prepared from a mesylate salt of abiraterone acetate.

5. The method of claim 1, wherein the therapeutically effective amount of the 17α-hydroxylase/C_{17,20}-lyase inhibitor is about 100 mg/day to about 2500 mg/day; about 250 mg/day to about 2000 mg/day; or about 1000 mg/day or more.

6. The method of claim 1, wherein the therapeutically effective amount of the 17α-hydroxylase/C_{17,20}-lyase inhibitor is administered at a frequency comprising (A) once per day; (B) once per day continuously during a first treatment cycle of about 28 days; (C) once per day continuously during a first treatment cycle of about 28 days, wherein the first treatment cycle is followed by a first non-treatment period during which the 17α-hydroxylase/C_{17,20}-lyase inhibitor is not administered to the patient; or (D) once per day continuously during a first treatment cycle of about 28 days, wherein the first treatment cycle is followed by a first non-treatment period during which the 17α-hydroxylase/C_{17,20}-lyase inhibitor is not administered to the patient, and wherein the first non-treatment period is followed by a second treatment cycle of about 28 days.

7. The method of claim 1, wherein the therapeutically effective amount of the 17α-hydroxylase/C_{17,20}-lyase inhibitor is administered orally.

8. The method of claim 1, wherein the therapeutically effective amount of the 17α-hydroxylase/C_{17,20}-lyase inhibitor is contained in a pharmaceutical composition that further comprises a pharmaceutically acceptable carrier or vehicle.

9. The method of claim 8, wherein the pharmaceutical composition is in the form of a tablet or a capsule.

10. The method of claim 1, further comprising administering the 17α-hydroxylase/C_{17,20}-lyase inhibitor (A) with food, (B) about 1 minute after consumption of food by the patient, (C) about 1 hour after consumption of food by the patient, or (D) once per day with food continuously during a first treatment course of about 28 days.

11. The method of claim 1, wherein the cancer is of the prostate, breast, ovary, testicle or other reproductive organ, bone, or lymph node.

12. The method of claim 1, wherein the cancer comprises an androgen-responsive cancer, a testosterone-responsive cancer, or an estrogen-responsive cancer.

13. The method of claim 1, wherein the cancer has metastasized or the cancer is a metastasis.

14. The method of claim 1, wherein the therapeutically effective amount of the 17α-hydroxylase/C_{17,20}-lyase inhibitor is administered while the patient is receiving another treatment comprising a chemotherapy or a hormone therapy.

15. The method of claim 1, wherein the patient is (A) about 30 to about 85 years old; or (B) about 65 years old or older.

16. The method of claim 1, wherein the patient that is refractory to a chemotherapy is hormone therapy-naïve.

17. The method of claim 1, wherein the patient has a baseline plasma concentration of PSA of about 0.5 ng/mL or more prior to administration of the therapeutically effective amount of the 17α-hydroxylase/C_{17,20}-lyase inhibitor.

18. The method of claim 17, wherein the patient has a baseline plasma concentration of PSA of about 4.0 ng/mL or more prior to administration of the therapeutically effective amount of the 17α-hydroxylase/C_{17,20}-lyase inhibitor.

19. The method of claim 1, wherein the patient has been castrated prior to administration of the therapeutically effective amount of the 17α-hydroxylase/C_{17,20}-lyase inhibitor.

20. The method of claim 1, wherein the patient has a first plasma concentration of PSA before administration of the therapeutically effective amount of the 17α-hydroxylase/C_{17,20}-lyase inhibitor and the patient has a second plasma concentration of PSA after administration of the therapeutically effective amount of the 17α-hydroxylase/C_{17,20}-lyase inhibitor, in which the second plasma concentration of PSA is less than the first plasma concentration of PSA.

21. The method of claim 20, wherein the second plasma concentration of PSA is less than the first plasma concentration of PSA for a time period of at least 10 days.

22. The method of claim 20, wherein the second plasma concentration of PSA is about 50 ng/mL or less.

23. The method of claim 20, wherein the second plasma concentration of PSA is about 50% or less of the first plasma concentration of PSA.

24. The method of claim 20, wherein the second plasma concentration of PSA is at or below castration levels of PSA.

25. The method of claim 24, wherein the second plasma concentration of PSA is at or below castration levels of PSA for a time period of at least 10 days.

26. The method of claim 1, wherein the patient has a first plasma concentration of testosterone before administration of the therapeutically effective amount of the 17α-hydroxylase/C_{17,20}-lyase inhibitor and the patient has a second plasma concentration of testosterone after administration of the therapeutically effective amount of the 17α-hydroxylase/C_{17,20}-lyase inhibitor, in which the second plasma concentration of testosterone is less than the first plasma concentration of testosterone.

27. The method of claim 26, wherein the second plasma concentration of testosterone is about 50% or less of the first plasma concentration of testosterone.

28. The method of claim 26, wherein the second plasma concentration of testosterone is at or below castration levels of testosterone.

29. The method of claim 28, wherein the second plasma concentration of testosterone is below castration levels of testosterone for a time period of at least 10 days.

30. The method of claim 1, wherein the patient has a first number of circulating tumor cells before administration of the therapeutically effective amount of the 17α-hydroxylase/C_{17,20}-lyase inhibitor and the patient has a second number of circulating tumor cells after administration of the therapeutically effective amount of the 17α-hydroxylase/C_{17,20}-lyase inhibitor, in which the second number of circulating tumor cells is fewer than the first number of circulating tumor cells.

31. The method of claim 30, wherein the second number of circulating tumor cells is about 75% or less of the first number of circulating tumor cells.

32. The method claim 1, wherein the cancer results in a tumor of a first tumor mass before administration of the therapeutically effective amount of the 17α-hydroxylase/C_{17,20}-lyase inhibitor, and wherein after administration of the therapeutically effective amount of the 17α-hydroxylase/C_{17,20}-lyase inhibitor the first tumor mass of the tumor is reduced to a second tumor mass that is smaller than the first tumor mass.

33. The method of claim 32, wherein the second tumor mass is about 50% or less of the first tumor mass.

34. The method of claim 1, wherein the cancer has metastasized to a bone or a lymph node before administration of the therapeutically effective amount of the 17α-hydroxylase/C_{17,20}-lyase inhibitor, and wherein the size of the metastasis in the patient is reduced or stabilized after administration of the therapeutically effective amount of the 17α-hydroxylase/C_{17,20}-lyase inhibitor.

35. The method of claim 34, wherein the size of the metastasis is reduced by 30% or more.

36. The method of claim 1, wherein the patient has a first plasma concentration of ALP before administration of the therapeutically effective amount of the 17α-hydroxylase/C_{17,20}-lyase inhibitor, and the patient has a second plasma concentration of ALP after administration of the therapeutically effective amount of the 17α-hydroxylase/C_{17,20}-lyase inhibitor, in which the second plasma concentration of ALP is less than the first plasma concentration of ALP.

37. The method of claim 36, wherein the second plasma concentration of ALP is about 30% or less of the first plasma concentration of ALP.

38. The method claim 1, wherein the patient experiences a first level of pain before administration of the therapeutically effective amount of the 17α-hydroxylase/C_{17,20}-lyase inhibitor, and the patient experiences a second level of pain after administration of the therapeutically effective amount of the 17α-hydroxylase/C_{17,20}-lyase inhibitor, in which the first level of pain is greater than the second level of pain.

39. The method of claim 1, wherein the patient was administered a first amount of a pain-relieving agent before administration of the therapeutically effective amount of the 17α-hydroxylase/C_{17,20}-lyase inhibitor, and the patient is administered a second amount of the pain-relieving agent after administration of the therapeutically effective amount of the 17α-hydroxylase/C_{17,20}-lyase inhibitor, in which the first amount of the pain-relieving agent is greater than the second amount of the pain-relieving agent.

40. The method of claim 1, wherein the therapeutically effective amount of the 17α-hydroxylase/C_{17,20}-lyase inhibitor provides a maximum plasma concentration of the 17α-hydroxylase/C_{17,20}-lyase inhibitor or a metabolite thereof, in the patient at about 1 hour to about 8 hours after administration of the therapeutically effective amount of the 17α-hydroxylase/C_{17,20}-lyase inhibitor.

41. The method of claim 40, wherein the maximum plasma concentration occurs at about 4 hours to about 6 hours after administration of the therapeutically effective amount of the 17α-hydroxylase/C_{17,20}-lyase inhibitor.

42. The method of claim 40, wherein the metabolite is abiraterone.

43. The method of claim 1, wherein the therapeutically effective amount of the 17α-hydroxylase/C_{17,20}-lyase inhibitor provides a maximum plasma concentration of the 17α-hydroxylase/C_{17,20}-lyase inhibitor, or a metabolite thereof, in the patient of about 50 nmol/L to about 5000 nmol/L.

44. The method of claim 43, wherein the maximum plasma concentration is about 250 nmol/L to about 4000 nmol/L.

45. The method of claim 43, wherein the maximum plasma concentration is about 200 nmol/L to about 2500 nmol/L.

46. The method of claim 43, wherein the metabolite is abiraterone.

47. The method of claim 1, wherein the therapeutically effective amount of the 17α-hydroxylase/C_{17,20}-lyase inhibitor provides an area under the curve of a plot of plasma concentration of the 17α-hydroxylase/C_{17,20}-lyase inhibitor, or a metabolite thereof, versus time of about 50 hr*nmol/L to about 25,000 hr*nmol/L.

48. The method of claim 47, wherein the area under the curve is about 1,000 hr*nmol/L to about 20,000 hr*nmol/L.

49. The method of claim 48, wherein the area under the curve is about 5,000 hr*nmol/L to about 18,000 hr*nmol/L.

50. A method for the treatment of a patient having cancer comprising administering to the patient having cancer that is (A) refractory to a hormone therapy, or (B) refractory to a hormone therapy and a chemotherapy, a therapeutically effective amount of a 17α-hydroxylase/C_{17,20}-lyase inhibitor with food.

51. The method of claim 50, wherein the 17α-hydroxylase/C_{17,20}-lyase inhibitor comprises a compound of the formula (I) or derivative, analog, pharmaceutically acceptable salt thereof 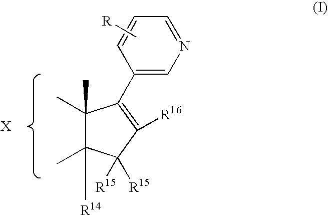
wherein X represents the residue of the A, B and C rings of a steroid which can be, without limitation, androstan-3α- or 3β-ol; androst-5-en-3α- or 3β-ol; androst-4-en-3-one; androst-2-ene; androst-4-ene; androst-5-ene; androsta-5,7-dien-3α or 3β-ol; androsta-1,4-dien-3-one; androsta-3,5-diene; androsta-3,5-diene-3-ol; estra-1,3,5[10]-triene; estra-1,3,5[10]-trien-3-ol; 5α-androstan-3-one; androst-4-ene-3,11-dione; 6-fluoroandrost-4-ene-3-one; or androstan-4-ene-3,6-dione; each of which, where structurally permissible, can be further derivatized in one or more of the following ways, including, but not limited to, to form 3-esters; to have one or more carbon or carbon ring double bonds in any of the 5,6-, 6,7-, 7,8-, 9,11- and 11,12-positions: as 3-oximes; as 3-methylenes; as 3-carboxylates; as 3-nitriles; as 3-nitros; as 3-desoxy derivatives; to have one or more hydroxy, halo, C₁₋₄-alkyl, trifluoro-methyl, C₁₋₄-alkoxy, C₁₋₄-alkanoyloxy, benzoyloxy, oxo, methylene or alkenyl substituents in the A, B, or C-ring; or to be 19-nor;
R represents a hydrogen atom or an alkyl group of 1-4 carbon atoms;
R¹⁴ represents a hydrogen atom, a halogen atom or an alkyl group of 1 to 4 carbon atoms;
each of the R¹⁵ substituents independently represents a hydrogen atom or an alkyl or alkoxy group of 1-4 carbon atoms, a hydroxy group or an alkylcarbonyloxy group of 2 to 5 carbon atoms or together represent an oxo or methylene group or R¹⁴ and one of the R¹⁵ groups together represent a double bond and the other R¹⁵ group represents a hydrogen atom or an alkyl group of 1 to 4 carbon atoms; and
R¹⁶ represents a hydrogen atom, halogen atom, or an alkyl group of 1 to 4 carbon atoms, in the form of the free bases or pharmaceutically acceptable acid addition salts, but excluding 3β-acetoxy-17-(3-pyridyl)androsta-5,14,16-triene, 3β,15α- and 3β,15β-diacetoxy-17-(3-pyridyl)androsta-5,16-diene and 3β-methoxy-17-(3-pyridyl-5α-androst-16-ene.

52. The method of claim 50, wherein the 17α-hydroxylase/C_{17,20}-lyase inhibitor comprises abiraterone acetate or a pharmaceutically acceptable salt thereof.

53. The method of claim 52, wherein the abiraterone acetate is prepared from a mesylate salt of abiraterone acetate.

54. The method of claim 50, wherein the therapeutically effective amount of the 17α-hydroxylase/C_{17,20}-lyase inhibitor is about 100 mg/day to about 2500 mg/day; about 250 mg/day to about 2000 mg/day; or about 1000 mg/day or more.

55. The method of claim 50, wherein the therapeutically effective amount of the 17α-hydroxylase/C_{17,20}-lyase inhibitor is administered at a frequency comprising (A) once per day; (B) once per day continuously during a first treatment cycle of about 28 days; (C) once per day continuously during a first treatment cycle of about 28 days, wherein the first treatment cycle is followed by a first non-treatment period during which the 17α-hydroxylase/C_{17,20}-lyase inhibitor is not administered to the patient; or (D) once per day continuously during a first treatment cycle of about 28 days, wherein the first treatment cycle is followed by a first non-treatment period during which the 17α-hydroxylase/C_{17,20}-lyase inhibitor is not administered to the patient, and wherein the first non-treatment period is followed by a second treatment cycle of about 28 days.

56. The method of claim 50, wherein the therapeutically effective amount of the 17α-hydroxylase/C_{17,20}-lyase inhibitor is administered orally.

57. The method of claim 50, wherein the therapeutically effective amount of the 17α-hydroxylase/C_{17,20}-lyase inhibitor is contained in a pharmaceutical composition that further comprises a pharmaceutically acceptable carrier or vehicle.

58. The method of claim 57, wherein the pharmaceutical composition is in the form of a tablet or a capsule.

59. The method of claim 50, further comprising administering the 17α-hydroxylase/C_{17,20}-lyase inhibitor once per day with food continuously during a first treatment course of about 28 days.

60. The method of claim 50, wherein the cancer is of the prostate, breast, ovary, testicle or other reproductive organ, bone, or lymph node.

61. The method of claim 50, wherein the cancer comprises an androgen-responsive cancer, a testosterone-responsive cancer, or an estrogen-responsive cancer.

62. The method of claim 50, wherein the cancer has metastasized or the cancer is a metastasis.

63. The method of claim 50, wherein the therapeutically effective amount of the 17α-hydroxylase/C_{17,20}-lyase inhibitor is administered while the patient is receiving another treatment comprising a chemotherapy or a hormone therapy.

64. The method of claim 50, wherein the patient is (A) about 30 to about 85 years old; or (B) about 65 years old or older.

65. The method of claim 50, wherein the patient that is refractory to a hormone therapy is chemotherapy-naïve.

66. The method of claim 50, wherein the patient has a baseline plasma concentration of PSA of about 0.5 ng/mL or more prior to administration of the therapeutically effective amount of the 17α-hydroxylase/C_{17,20}-lyase inhibitor.

67. The method of claim 66, wherein the patient has a baseline plasma concentration of PSA of about 4.0 ng/mL or more prior to administration of the therapeutically effective amount of the 17α-hydroxylase/C_{17,20}-lyase inhibitor.

68. The method of claim 50, wherein the patient has been castrated prior to administration of the therapeutically effective amount of the 17α-hydroxylase/C_{17,20}-lyase inhibitor.

69. The method of claim 50, wherein the patient has a first plasma concentration of PSA before administration of the therapeutically effective amount of the 17α-hydroxylase/C_{17,20}-lyase inhibitor and the patient has a second plasma concentration of PSA after administration of the therapeutically effective amount of the 17α-hydroxylase/C_{17,20}-lyase inhibitor, in which the second plasma concentration of PSA is less than the first plasma concentration of PSA.

70. The method of claim 69, wherein the second plasma concentration of PSA is less than the first plasma concentration of PSA for a time period of at least 10 days.

71. The method of claim 69, wherein the second plasma concentration of PSA is about 50 ng/mL or less.

72. The method of claim 69, wherein the second plasma concentration of PSA is about 50% or less of the first plasma concentration of PSA.

73. The method of claim 69, wherein the second plasma concentration of PSA is at or below castration levels of PSA.

74. The method of claim 73, wherein the second plasma concentration of PSA is at or below castration levels of PSA for a time period of at least 10 days.

75. The method of claim 50, wherein the patient has a first plasma concentration of testosterone before administration of the therapeutically effective amount of the 17α-hydroxylase/C_{17,20}-lyase inhibitor and the patient has a second plasma concentration of testosterone after administration of the therapeutically effective amount of the 17α-hydroxylase/C_{17,20}-lyase inhibitor, in which the second plasma concentration of testosterone is less than the first plasma concentration of testosterone.

76. The method of claim 75, wherein the second plasma concentration of testosterone is about 50% or less of the first plasma concentration of testosterone.

77. The method of claim 75, wherein the second plasma concentration of testosterone is at or below castration levels of testosterone.

78. The method of claim 77, wherein the second plasma concentration of testosterone is below castration levels of testosterone for a time period of at least 10 days.

79. The method of claim 50, wherein the patient has a first number of circulating tumor cells before administration of the therapeutically effective amount of the 17α-hydroxylase/C_{17,20}-lyase inhibitor and the patient has a second number of circulating tumor cells after administration of the therapeutically effective amount of the 17α-hydroxylase/C_{17,20}-lyase inhibitor, in which the second number of circulating tumor cells is fewer than the first number of circulating tumor cells.

80. The method of claim 79, wherein the second number of circulating tumor cells is about 75% or less of the first number of circulating tumor cells.

81. The method claim 50, wherein the cancer results in a tumor of a first tumor mass before administration of the therapeutically effective amount of the 17α-hydroxylase/C_{17,20}-lyase inhibitor, and wherein after administration of the therapeutically effective amount of the 17α-hydroxylase/C_{17,20}-lyase inhibitor the first tumor mass of the tumor is reduced to a second tumor mass that is smaller than the first tumor mass.

82. The method of claim 81, wherein the second tumor mass is about 50% or less of the first tumor mass.

83. The method of claim 50, wherein the cancer has metastasized to a bone or a lymph node before administration of the therapeutically effective amount of the 17α-hydroxylase/C₁₇₋₂₀-lyase inhibitor, and wherein the size of the metastasis in the patient is reduced or stabilized after administration of the therapeutically effective amount of the 17α-hydroxylase/C_{17,20}-lyase inhibitor.

84. The method of claim 83, wherein the size of the metastasis is reduced by 30% or more.

85. The method of claim 50, wherein the patient has a first plasma concentration of ALP before administration of the therapeutically effective amount of the 17α-hydroxylase/C_{17,20}-lyase inhibitor, and the patient has a second plasma concentration of ALP after administration of the therapeutically effective amount of the 17α-hydroxylase/C_{17,20}-lyase inhibitor, in which the second plasma concentration of ALP is less than the first plasma concentration of ALP.

86. The method of claim 85, wherein the second plasma concentration of ALP is about 30% or less of the first plasma concentration of ALP.

87. The method claim 50, wherein the patient experiences a first level of pain before administration of the therapeutically effective amount of the 17α-hydroxylase/C_{17,20}-lyase inhibitor, and the patient experiences a second level of pain after administration of the therapeutically effective amount of the 17α-hydroxylase/C_{17,20}-lyase inhibitor, in which the first level of pain is greater than the second level of pain.

88. The method of claim 50, wherein the patient was administered a first amount of a pain-relieving agent before administration of the therapeutically effective amount of the 17α-hydroxylase/C_{17,20}-lyase inhibitor, and the patient is administered a second amount of the pain-relieving agent after administration of the therapeutically effective amount of the 17α-hydroxylase/C_{17,20}-lyase inhibitor, in which the first amount of the pain-relieving agent is greater than the second amount of the pain-relieving agent.

89. The method of claim 50, wherein the therapeutically effective amount of the 17α-hydroxylase/C_{17,20}-lyase inhibitor provides a maximum plasma concentration of the 17α-hydroxylase/C_{17,20}-lyase inhibitor, or a metabolite thereof, in the patient at about 1 hour to about 8 hours after administration of the therapeutically effective amount of the 17α-hydroxylase/C_{17,20}-lyase inhibitor.

90. The method of claim 89, wherein the maximum plasma concentration occurs at about 4 hours to about 6 hours after administration of the therapeutically effective amount of the 17α-hydroxylase/C_{17,20}-lyase inhibitor.

91. The method of claim 89, wherein the metabolite is abiraterone.

92. The method of claim 50, wherein the therapeutically effective amount of the 17α-hydroxylase/C_{17,20}-lyase inhibitor provides a maximum plasma concentration of the 17α-hydroxylase/C_{17,20}-lyase inhibitor, or a metabolite thereof, in the patient of about 50 nmol/L to about 5000 nmol/L.

93. The method of claim 92, wherein the maximum plasma concentration is about 250 nmol/L to about 4000 nmol/L.

94. The method of claim 92, wherein the maximum plasma concentration is about 200 nmol/L to about 2500 nmol/L.

95. The method of claim 92, wherein the metabolite is abiraterone.

96. The method of claim 50, wherein the therapeutically effective amount of the 17α-hydroxylase/C_{17,20}-lyase inhibitor provides an area under the curve of a plot of plasma concentration of the 17α-hydroxylase/C_{17,20}-lyase inhibitor, or a metabolite thereof, versus time of about 50 hr*nmol/L to about 25,000 hr*nmol/L.

97. The method of claim 96, wherein the area under the curve is about 1,000 hr*nmol/L to about 20,000 hr*nmol/L.

98. The method of claim 97, wherein the area under the curve is about 5,000 hr*nmol/L to about 18,000 hr*nmol/L.

99. A method for the treatment of a patient having cancer comprising administering to the patient having cancer a therapeutically effective amount of a 17α-hydroxylase/C_{17,20}-lyase inhibitor comprising about 825 mg/day or more.

100. The method of claim 99, wherein the therapeutically effective amount of the 17α-hydroxylase/C_{17,20}-lyase inhibitor comprises about 1000 mg/day or more.

101. The method of claim 99, wherein the 17α-hydroxylase/C_{17,20}-lyase inhibitor comprises a compound of the formula (I) or derivative, analog, pharmaceutically acceptable salt thereof 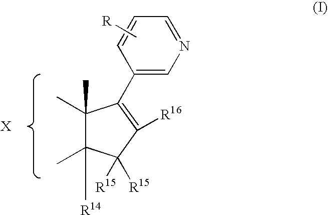
wherein X represents the residue of the A, B and C rings of a steroid which can be, without limitation, androstan-3α- or 3β-ol; androst-5-en-3α- or 3β-ol; androst-4-en-3-one; androst-2-ene; androst-4-ene; androst-5-ene; androsta-5,7-dien-3α or 3β-ol; androsta-1,4-dien-3-one; androsta-3,5-diene; androsta-3,5-diene-3-ol; estra-1,3,5[10]-triene; estra-1,3,5[10]-trien-3-ol; 5α-androstan-3-one; androst-4-ene-3,11-dione; 6-fluoroandrost-4-ene-3-one; or androstan-4-ene-3,6-dione; each of which, where structurally permissible, can be further derivatized in one or more of the following ways, including, but not limited to, to form 3-esters; to have one or more carbon or carbon ring double bonds in any of the 5,6-, 6,7-, 7,8-, 9,11- and 11,12-positions; as 3-oximes; as 3-methylenes; as 3-carboxylates; as 3-nitriles; as 3-nitros; as 3-desoxy derivatives; to have one or more hydroxy, halo, C₁₋₄-alkyl, trifluoro-methyl, C₁₋₄-alkoxy, C₁₋₄-alkanoyloxy, benzoyloxy, oxo, methylene or alkenyl substituents in the A, B, or C-ring; or to be 19-nor;
R represents a hydrogen atom or an alkyl group of 1-4 carbon atoms;
R¹⁴ represents a hydrogen atom, a halogen atom or an alkyl group of 1 to 4 carbon atoms;
each of the R¹⁵ substituents independently represents a hydrogen atom or an alkyl or alkoxy group of 1-4 carbon atoms, a hydroxy group or an alkylcarbonyloxy group of 2 to 5 carbon atoms or together represent an oxo or methylene group or R¹⁴ and one of the R¹⁵ groups together represent a double bond and the other R¹⁵ group represents a hydrogen atom or an alkyl group of 1 to 4 carbon atoms; and
R¹⁶ represents a hydrogen atom, halogen atom, or an alkyl group of 1 to 4 carbon atoms,
in the form of the free bases or pharmaceutically acceptable acid addition salts, but excluding 3β-acetoxy-17-(3-pyridyl)androsta-5,14,16-triene, 3β,15α- and 3β,15β-diacetoxy-17-(3-pyridyl)androsta-5,16-diene and 3β-methoxy-17-(3-pyridyl-5α-androst-16-ene.

102. The method of claim 99, wherein the 17α-hydroxylase/C_{17,20}-lyase inhibitor comprises abiraterone acetate or a pharmaceutically acceptable salt thereof.

103. The method of claim 102, wherein the abiraterone acetate is prepared from a mesylate salt of abiraterone acetate.

104. The method of claim 99, wherein the therapeutically effective amount of the 17α-hydroxylase/C_{17,20}-lyase inhibitor is administered at a frequency comprising (A) once per day; (B) once per day continuously during a first treatment cycle of about 28 days; (C) once per day continuously during a first treatment cycle of about 28 days, wherein the first treatment cycle is followed by a first non-treatment period during which the 17α-hydroxylase/C_{17,20}-lyase inhibitor is not administered to the patient; or (D) once per day continuously during a first treatment cycle of about 28 days, wherein the first treatment cycle is followed by a first non-treatment period during which the 17α-hydroxylase/C_{17,20}-lyase inhibitor is not administered to the patient, and wherein the first non-treatment period is followed by a second treatment cycle of about 28 days.

105. The method of claim 99, wherein the therapeutically effective amount of the 17α-hydroxylase/C_{17,20}-lyase inhibitor is administered orally.

106. The method of claim 99, wherein the therapeutically effective amount of the 17α-hydroxylase/C_{17,20}-lyase inhibitor is contained in a pharmaceutical composition that further comprises a pharmaceutically acceptable carrier or vehicle.

107. The method of claim 106, wherein the pharmaceutical composition is in the form of a tablet or a capsule.

108. The method of claim 99, further comprising administering the 17α-hydroxylase/C_{17,20}-lyase inhibitor (A) with food, (B) about 1 minute after consumption of food by the patient, (C) about 1 hour after consumption of food by the patient, or (D) once per day with food continuously during a first treatment course of about 28 days.

109. The method of claim 99, wherein the cancer is of the prostate, breast, ovary, testicle or other reproductive organ, bone, or lymph node.

110. The method of claim 99, wherein the cancer comprises an androgen-responsive cancer, a testosterone-responsive cancer, or an estrogen-responsive cancer.

111. The method of claim 99, wherein the cancer has metastasized or the cancer is a metastasis.

112. The method of claim 99, wherein the therapeutically effective amount of the 17α-hydroxylase/C_{17,20}-lyase inhibitor is administered while the patient is receiving another treatment comprising a chemotherapy or a hormone therapy.

113. The method of claim 99, wherein the patient is (A) about 30 to about 85 years old; or (B) about 65 years old or older.

114. The method of claim 99, wherein the patient is chemotherapy-naïve.

115. The method of claim 99, wherein the patient is hormone therapy-naïve.

116. The method of claim 99, wherein the patient has a baseline plasma concentration of PSA of about 0.5 ng/mL or more prior to administration of the therapeutically effective amount of the 17α-hydroxylase/C_{17,20}-lyase inhibitor.

117. The method of claim 116, wherein the patient has a baseline plasma concentration of PSA of about 4.0 ng mL or more prior to administration of the therapeutically effective amount of the 17α-hydroxylase/C_{17,20}-lyase inhibitor.

118. The method of claim 99, wherein the patient has been castrated prior to administration of the therapeutically effective amount of the 17α-hydroxylase/C_{17,20}-lyase inhibitor.

119. The method of claim 99, wherein the patient has a first plasma concentration of PSA before administration of the therapeutically effective amount of the 17α-hydroxylase/C_{17,20}-lyase inhibitor and the patient has a second plasma concentration of PSA after administration of the therapeutically effective amount of the 17α-hydroxylase/C_{17,20}-lyase inhibitor, in which the second plasma concentration of PSA is less than the first plasma concentration of PSA.

120. The method of claim 119, wherein the second plasma concentration of PSA is less than the first plasma concentration of PSA for a time period of at least 10 days.

121. The method of claim 119, wherein the second plasma concentration of PSA is about 50 ng/mL or less.

122. The method of claim 119, wherein the second plasma concentration of PSA is about 50% or less of the first plasma concentration of PSA.

123. The method of claim 119, wherein the second plasma concentration of PSA is at or below castration levels of PSA.

124. The method of claim 123, wherein the second plasma concentration of PSA is at or below castration levels of PSA for a time period of at least 10 days.

125. The method of claim 99, wherein the patient has a first plasma concentration of testosterone before administration of the therapeutically effective amount of the 17α-hydroxylase/C_{17,20}-lyase inhibitor and the patient has a second plasma concentration of testosterone after administration of the therapeutically effective amount of the 17α-hydroxylase/C_{17,20}-lyase inhibitor, in which the second plasma concentration of testosterone is less than the first plasma concentration of testosterone.

126. The method of claim 125, wherein the second plasma concentration of testosterone is about 50% or less of the first plasma concentration of testosterone.

127. The method of claim 125, wherein the second plasma concentration of testosterone is at or below castration levels of testosterone.

128. The method of claim 127, wherein the second plasma concentration of testosterone is below castration levels of testosterone for a time period of at least 10 days.

129. The method of claim 99, wherein the patient has a first number of circulating tumor cells before administration of the therapeutically effective amount of the 17α-hydroxylase/C_{17,20}-lyase inhibitor and the patient has a second number of circulating tumor cells after administration of the therapeutically effective amount of the 17α-hydroxylase/C_{17,20}-lyase inhibitor, in which the second number of circulating tumor cells is fewer than the first number of circulating tumor cells.

130. The method of claim 129, wherein the second number of circulating tumor cells is about 75% or less of the first number of circulating tumor cells.

131. The method claim 99, wherein the cancer results in a tumor of a first tumor mass before administration of the therapeutically effective amount of the 17α-hydroxylase/C_{17,20}-lyase inhibitor, and wherein after administration of the therapeutically effective amount of the 17α-hydroxylase/C_{17,20}-lyase inhibitor the first tumor mass of the tumor is reduced to a second tumor mass that is smaller than the first tumor mass.

132. The method of claim 131, wherein the second tumor mass is about 50% or less of the first tumor mass.

133. The method of claim 99, wherein the cancer has metastasized to a bone or a lymph node before administration of the therapeutically effective amount of the 17α-hydroxylase/C_{17,20}-lyase inhibitor, and wherein the size of the metastasis in the patient is reduced or stabilized after administration of the therapeutically effective amount of the 17α-hydroxylase/C_{17,20}-lyase inhibitor.

134. The method of claim 133, wherein the size of the metastasis is reduced by 30% or more.

135. The method of claim 99, wherein the patient has a first plasma concentration of ALP before administration of the therapeutically effective amount of the 17α-hydroxylase/C_{17,20}-lyase inhibitor, and the patient has a second plasma concentration of ALP after administration of the therapeutically effective amount of the 17α-hydroxylase/C_{17,20}-lyase inhibitor, in which the second plasma concentration of ALP is less than the first plasma concentration of ALP.

136. The method of claim 135, wherein the second plasma concentration of ALP is about 30% or less of the first plasma concentration of ALP.

137. The method claim 99, wherein the patient experiences a first level of pain before administration of the therapeutically effective amount of the 177α-hydroxylase/C_{17,20}-lyase inhibitor, and the patient experiences a second level of pain after administration of the therapeutically effective amount of the 17α-hydroxylase/C_{17,20}-lyase inhibitor, in which the first level of pain is greater than the second level of pain.

138. The method of claim 99, wherein the patient was administered a first amount of a pain-relieving agent before administration of the therapeutically effective amount of the 17α-hydroxylase/C_{17,20}-lyase inhibitor, and the patient is administered a second amount of the pain-relieving agent after administration of the therapeutically effective amount of the 17α-hydroxylase/C_{17,20}-lyase inhibitor, in which the first amount of the pain-relieving agent is greater than the second amount of the pain-relieving agent.

139. The method of claim 99, wherein the therapeutically effective amount of the 17α-hydroxylase/C_{17,20}-lyase inhibitor provides a maximum plasma concentration of the 17α-hydroxylase/C_{17,20}-lyase inhibitor, or a metabolite thereof, in the patient at about 1 hour to about 8 hours after administration of the therapeutically effective amount of the 17α-hydroxylase/C_{17,20}-lyase inhibitor.

140. The method of claim 139, wherein the maximum plasma concentration occurs at about 4 hours to about 6 hours after administration of the therapeutically effective amount of the 17α-hydroxylase/C_{17,20}-lyase inhibitor.

141. The method of claim 139, wherein the metabolite is abiraterone.

142. The method of claim 99, wherein the therapeutically effective amount of the 17α-hydroxylase/C_{17,20}-lyase inhibitor provides a maximum plasma concentration of the 17α-hydroxylase/C_{17,20}-lyase inhibitor, or a metabolite thereof, in the patient of about 50 nmol/L to about 5000 nmol/L.

143. The method of claim 142, wherein the maximum plasma concentration is about 250 nmol/L to about 4000 nmol/L.

144. The method of claim 142, wherein the maximum plasma concentration is about 200 nmol/L to about 2500 nmol/L.

145. The method of claim 142, wherein the metabolite is abiraterone.

146. The method of claim 99, wherein the therapeutically effective amount of the 17α-hydroxylase/C_{17,20}-lyase inhibitor provides an area under the curve of a plot of plasma concentration of the 17α-hydroxylase/C_{17,20}-lyase inhibitor, or a metabolite thereof, versus time of about 50 hr*nmol/L to about 25,000 hr*nmol/L.

147. The method of claim 146, wherein the area under the curve is about 1,000 hr*nmol/L to about 20,000 hr*nmol/L.

148. The method of claim 147, wherein the area under the curve is about 5,000 hr*nmol/L to about 18,000 hr*nmol/L.

149. A method for the treatment of a patient having cancer comprising administering to the patient having cancer a therapeutically effective amount of a 17α-hydroxylase/C_{17,20}-lyase inhibitor with food.

150. The method of claim 149, wherein the 17α-hydroxylase/C_{17,20}-lyase inhibitor comprises a compound of the formula (I) or derivative, analog, pharmaceutically acceptable salt thereof 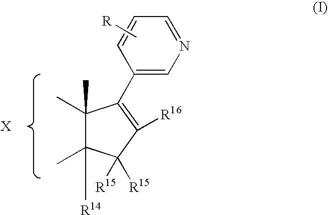
wherein X represents the residue of the A, B and C rings of a steroid which can be, without limitation, androstan-3α- or 3β-ol; androst-5-en-3α- or 3β-ol; androst-4-en-3-one; androst-2-ene; androst-4-ene; androst-5-ene; androsta-5,7-dien-3α or 3β-ol; androsta-1,4-dien-3-one; androsta-3,5-diene; androsta-3,5-diene-3-ol; estra-1,3,5[10]-triene; estra-1,3,5[10]-trien-3-ol; 5α-androstan-3-one; androst-4-ene-3,11-dione; 6-fluoroandrost-4-ene-3-one; or androstan-4-ene-3,6-dione; each of which, where structurally permissible, can be further derivatized in one or more of the following ways, including, but not limited to, to form 3-esters; to have one or more carbon or carbon ring double bonds in any of the 5,6-, 6,7-, 7,8-, 9,11- and 11,12-positions; as 3-oximes; as 3-methylenes; as 3-carboxylates; as 3-nitriles; as 3-nitros; as 3-desoxy derivatives; to have one or more hydroxy, halo, C₁₋₄-alkyl, trifluoro-methyl, C₁₋₄-alkoxy, C₁₋₄-alkanoyloxy, benzoyloxy, oxo, methylene or alkenyl substituents in the A, B, or C-ring; or to be 19-nor;
R represents a hydrogen atom or an alkyl group of 1-4 carbon atoms;
R¹⁴ represents a hydrogen atom, a halogen atom or an alkyl group of 1 to 4 carbon atoms;
each of the R¹⁵ substituents independently represents a hydrogen atom or an alkyl or alkoxy group of 1-4 carbon atoms, a hydroxy group or an alkylcarbonyloxy group of 2 to 5 carbon atoms or together represent an oxo or methylene group or R¹⁴ and one of the R¹⁵ groups together represent a double bond and the other R¹⁵ group represents a hydrogen atom or an alkyl group of 1 to 4 carbon atoms; and
R¹⁶ represents a hydrogen atom, halogen atom, or an alkyl group of 1 to 4 carbon atoms,
in the form of the free bases or pharmaceutically acceptable acid addition salts, but excluding 3β-acetoxy-17-(3-pyridyl)androsta-5,14,16-triene, 3β, 15α- and 3β,15β-diacetoxy-17-(3-pyridyl)androsta-5,16-diene and 3β-methoxy-17-(3-pyridyl-5α-androst-16-ene.

151. The method of claim 149, wherein the 17α-hydroxylase/C_{17,20}-lyase inhibitor comprises abiraterone acetate or a pharmaceutically acceptable salt thereof.

152. The method of claim 151, wherein the abiraterone acetate is prepared from a mesylate salt of abiraterone acetate.

153. The method of claim 149, wherein the therapeutically effective amount of the 17α-hydroxylase/C_{17,20}-lyase inhibitor is about 100 mg/day to about 2500 mg/day; about 250 mg/day to about 2000 mg/day; or about 1000 mg/day or more.

154. The method of claim 149, wherein the therapeutically effective amount of the 17α-hydroxylase/C_{17,20}-lyase inhibitor is administered at a frequency comprising (A) once per day; (B) once per day continuously during a first treatment cycle of about 28 days; (C) once per day continuously during a first treatment cycle of about 28 days, wherein the first treatment cycle is followed by a first non-treatment period during which the 17α-hydroxylase/C_{17,20}-lyase inhibitor is not administered to the patient; or (D) once per day continuously during a first treatment cycle of about 28 days, wherein the first treatment cycle is followed by a first non-treatment period during which the 17α-hydroxylase/C_{17,20}-lyase inhibitor is not administered to the patient, and wherein the first non-treatment period is followed by a second treatment cycle of about 28 days.

155. The method of claim 149, wherein the therapeutically effective amount of the 17α-hydroxylase/C_{17,20}-lyase inhibitor is administered orally.

156. The method of claim 149, wherein the therapeutically effective amount of the 17α-hydroxylase/C_{17,20}-lyase inhibitor is contained in a pharmaceutical composition that further comprises a pharmaceutically acceptable carrier or vehicle.

157. The method of claim 156, wherein the pharmaceutical composition is in the form of a tablet or a capsule.

158. The method of claim 149, further comprising administering the 17α-hydroxylase/C_{17,20}-lyase inhibitor once per day with food continuously during a first treatment course of about 28 days.

159. The method of claim 149, wherein the cancer is of the prostate, breast, ovary, testicle or other reproductive organ, bone, or lymph node.

160. The method of claim 149, wherein the cancer comprises an androgen-responsive cancer, a testosterone-responsive cancer, or an estrogen-responsive cancer.

161. The method of claim 149, wherein the cancer has metastasized or the cancer is a metastasis.

162. The method of claim 149, wherein the therapeutically effective amount of the 17α-hydroxylase/C_{17,20}-lyase inhibitor is administered while the patient is receiving another treatment comprising a chemotherapy or a hormone therapy.

163. The method of claim 149, wherein the patient is (A) about 30 to about 85 years old; or (B) about 65 years old or older.

164. The method of claim 149, wherein the patient is chemotherapy-naïve.

165. The method of claim 149, wherein the patient is hormone therapy-naïve.

166. The method of claim 149, wherein the patient has a baseline plasma concentration of PSA of about 0.5 ng/mL or more prior to administration of the therapeutically effective amount of the 17α-hydroxylase/C_{17,20}-lyase inhibitor.

167. The method of claim 166, wherein the patient has a baseline plasma concentration of PSA of about 4.0 ng/mL or more prior to administration of the therapeutically effective amount of the 17α-hydroxylase/C_{17,20}-lyase inhibitor.

168. The method of claim 149, wherein the patient has been castrated prior to administration of the therapeutically effective amount of the 17-hydroxylase/C_{17,20}-lyase inhibitor.

169. The method of claim 149, wherein the patient has a first plasma concentration of PSA before administration of the therapeutically effective amount of the 17α-hydroxylase/C_{17,20}-lyase inhibitor and the patient has a second plasma concentration of PSA after administration of the therapeutically effective amount of the 17-hydroxylase/C_{17,20}-lyase inhibitor, in which the second plasma concentration of PSA is less than the first plasma concentration of PSA.

170. The method of claim 169, wherein the second plasma concentration of PSA is less than the first plasma concentration of PSA for a time period of at least 10 days.

171. The method of claim 169, wherein the second plasma concentration of PSA is about 50 ng/mL or less.

172. The method of claim 169, wherein the second plasma concentration of PSA is about 50% or less of the first plasma concentration of PSA.

173. The method of claim 169, wherein the second plasma concentration of PSA is at or below castration levels of PSA.

174. The method of claim 173, wherein the second plasma concentration of PSA is at or below castration levels of PSA for a time period of at least 10 days.

175. The method of claim 149, wherein the patient has a first plasma concentration of testosterone before administration of the therapeutically effective amount of the 17α-hydroxylase/C_{17,20}-lyase inhibitor and the patient has a second plasma concentration of testosterone after administration of the therapeutically effective amount of the 17α-hydroxylase/C_{17,20}-lyase inhibitor, in which the second plasma concentration of testosterone is less than the first plasma concentration of testosterone.

176. The method of claim 175, wherein the second plasma concentration of testosterone is about 50% or less of the first plasma concentration of testosterone.

177. The method of claim 175, wherein the second plasma concentration of testosterone is at or below castration levels of testosterone.

178. The method of claim 177, wherein the second plasma concentration of testosterone is below castration levels of testosterone for a time period of at least 10 days.

179. The method of claim 149, wherein the patient has a first number of circulating tumor cells before administration of the therapeutically effective amount of the 17α-hydroxylase/C_{17,20}-lyase inhibitor and the patient has a second number of circulating tumor cells after administration of the therapeutically effective amount of the 17α-hydroxylase/C_{17,20}-lyase inhibitor, in which the second number of circulating tumor cells is fewer than the first number of circulating tumor cells.

180. The method of claim 179, wherein the second number of circulating tumor cells is about 75% or less of the first number of circulating tumor cells.

181. The method claim 149, wherein the cancer results in a tumor of a first tumor mass before administration of the therapeutically effective amount of the 17α-hydroxylase/C_{17,20}-lyase inhibitor, and wherein after administration of the therapeutically effective amount of the 17α-hydroxylase/C_{17,20}-lyase inhibitor the first tumor mass of the tumor is reduced to a second tumor mass that is smaller than the first tumor mass.

182. The method of claim 181, wherein the second tumor mass is about 50% or less of the first tumor mass.

183. The method of claim 149, wherein the cancer has metastasized to a bone or a lymph node before administration of the therapeutically effective amount of the 17α-hydroxylase/C_{17,20}-lyase inhibitor, and wherein the size of the metastasis in the patient is reduced or stabilized after administration of the therapeutically effective amount of the 17α-hydroxylase/C_{17,20}-lyase inhibitor.

184. The method of claim 183, wherein the size of the metastasis is reduced by 30% or more.

185. The method of claim 149, wherein the patient has a first plasma concentration of ALP before administration of the therapeutically effective amount of the 17α-hydroxylase/C_{17,20}-lyase inhibitor, and the patient has a second plasma concentration of ALP after administration of the therapeutically effective amount of the 17α-hydroxylase/C_{17,20}-lyase inhibitor, in which the second plasma concentration of ALP is less than the first plasma concentration of ALP.

186. The method of claim 185, wherein the second plasma concentration of ALP is about 30% or less of the first plasma concentration of ALP.

187. The method claim 149, wherein the patient experiences a first level of pain before administration of the therapeutically effective amount of the 17α-hydroxylase/C_{17,20}-lyase inhibitor, and the patient experiences a second level of pain after administration of the therapeutically effective amount of the 17α-hydroxylase/C_{17,20}-lyase inhibitor, in which the first level of pain is greater than the second level of pain.

188. The method of claim 149, wherein the patient was administered a first amount of a pain-relieving agent before administration of the therapeutically effective amount of the 17α-hydroxylase/C_{17,20}-lyase inhibitor, and the patient is administered a second amount of the pain-relieving agent after administration of the therapeutically effective amount of the 17α-hydroxylase/C_{17,20}-lyase inhibitor, in which the first amount of the pain-relieving agent is greater than the second amount of the pain-relieving agent.

189. The method of claim 149, wherein the therapeutically effective amount of the 17α-hydroxylase/C_{17,20}-lyase inhibitor provides a maximum plasma concentration of the 17α-hydroxylase/C_{17,20}-lyase inhibitor, or a metabolite thereof, in the patient at about 1 hour to about 8 hours after administration of the therapeutically effective amount of the 17α-hydroxylase/C_{17,20}-lyase inhibitor.

190. The method of claim 189, wherein the maximum plasma concentration occurs at about 4 hours to about 6 hours after administration of the therapeutically effective amount of the 17α-hydroxylase/C_{17,20}-lyase inhibitor.

191. The method of claim 189, wherein the metabolite is abiraterone.

192. The method of claim 149, wherein the therapeutically effective amount of the 17α-hydroxylase/C_{17,20}-lyase inhibitor provides a maximum plasma concentration of the 17α-hydroxylase/C_{17,20}-lyase inhibitor, or a metabolite thereof, in the patient of about 50 nmol/L to about 5000 nmol/L.

193. The method of claim 192, wherein the maximum plasma concentration is about 250 nmol/L to about 4000 nmol/L.

194. The method of claim 192, wherein the maximum plasma concentration is about 200 nmol/L to about 2500 nmol/L.

195. The method of claim 192, wherein the metabolite is abiraterone.

196. The method of claim 149, wherein the therapeutically effective amount of the 17α-hydroxylase/C_{17,20}-lyase inhibitor provides an area under the curve of a plot of plasma concentration of the 17α-hydroxylase/C_{17,20}-lyase inhibitor, or a metabolite thereof, versus time of about 50 hr*nmol/L to about 25,000 hr*nmol/L.

197. The method of claim 196, wherein the area under the curve is about 1,000 hr*nmol/L to about 20,000 hr*nmol/L.

198. The method of claim 197, wherein the area under the curve is about 5,000 hr*nmol/L to about 18,000 hr*nmol/L.

199. A method for the treatment of a patient having prostate cancer comprising administering to the patient having prostate cancer that is refractory to a chemotherapy a therapeutically effective amount of abiraterone acetate, wherein the therapeutically effective amount is about 800 mg/day to about 2,000 mg/day, and wherein the maximum plasma concentration of the abiraterone acetate or a metabolite thereof in the patient is about 50 nmol/L to about 5000 nmol/L.

200. A method for the treatment of a patient having prostate cancer comprising administering to the patient having prostate cancer that is refractory to a hormone therapy a therapeutically effective amount of abiraterone acetate, wherein the therapeutically effective amount is about 800 mg/day to about 2,000 mg/day, and wherein the maximum plasma concentration of the abiraterone acetate or a metabolite thereof in the patient is about 50 nmol/L to about 5000 nmol/L.

201. A method for the treatment of a patient having prostate cancer comprising administering to the patient having prostate cancer that is refractory to a chemotherapy and refractory to a hormone therapy a therapeutically effective amount of abiraterone acetate, wherein the therapeutically effective amount is about 800 mg/day to about 2,000 mg/day, and wherein the maximum plasma concentration of the abiraterone acetate or a metabolite thereof in the patient is about 50 nmol/L to about 5000 nmol/L.

202. A method for the treatment of a patient having prostate cancer comprising administering to the patient having prostate cancer that is refractory to a chemotherapy a therapeutically effective amount of abiraterone acetate, wherein the therapeutically effective amount is about 800 mg/day to about 2,000 mg/day, and wherein the maximum plasma concentration of abiraterone acetate or a metabolite thereof in the patient occurs at about 1 hour to about 8 hours after administration of the therapeutically effective amount of abiraterone acetate.

203. A method for the treatment of a patient having prostate cancer comprising administering to the patient having prostate cancer that is refractory to a hormone therapy a therapeutically effective amount of abiraterone acetate, wherein the therapeutically effective amount is about 800 mg/day to about 2,000 mg/day, and wherein the maximum plasma concentration of abiraterone acetate or a metabolite thereof in the patient occurs at about 1 hour to about 8 hours after administration of the therapeutically effective amount of abiraterone acetate.

204. A method for the treatment of a patient having prostate cancer comprising administering to the patient having prostate cancer that is refractory to a chemotherapy and refractory to a hormone therapy a therapeutically effective amount of abiraterone acetate, wherein the therapeutically effective amount is about 800 mg/day to about 2,000 mg/day, and wherein the maximum plasma concentration of abiraterone acetate or a metabolite thereof in the patient occurs at about 1 hour to about 8 hours after administration of the therapeutically effective amount of abiraterone acetate.

205. A method for the treatment of a patient having prostate cancer comprising administering to the patient having prostate cancer that is refractory to a chemotherapy a therapeutically effective amount of abiraterone acetate, wherein the patient has been castrated prior to administration of the therapeutically effective amount of abiraterone acetate, and wherein the prostate cancer has metastasized.

206. A method for the treatment of a patient having prostate cancer comprising administering to the patient having prostate cancer that is refractory to a hormone therapy a therapeutically effective amount of abiraterone acetate, wherein the patient has been castrated prior to administration of the therapeutically effective amount of abiraterone acetate, and wherein the prostate cancer has metastasized.

207. A method for the treatment of a patient having prostate cancer comprising administering to the patient having prostate cancer that is refractory to a chemotherapy and refractory to a hormone therapy a therapeutically effective amount of abiraterone acetate, wherein the patient has been castrated prior to administration of the therapeutically effective amount of abiraterone acetate, and wherein the prostate cancer has metastasized.
